(19) United States
(12) Patent Application Publication
(10) Pub. No.: US 2020/0399631 A1
(43) Pub. Date: Dec. 24, 2020

(54) Methods and Compositions for Sample Analysis

(71) Applicant: 10X Genomics, Inc., Pleasanton, CA (US)

(72) Inventors: Mirna Jarosz, Mountain View, CA (US); Christopher Hindson, Pleasanton, CA (US); Michael Schnall-Levin, Palo Alto, CA (US); Kevin Dean Ness, Pleasanton, CA (US); Serge Saxonov, Oakland, CA (US); Benjamin Hindson, Pleasanton, CA (US); John Stuelpnagel, Pleasanton, CA (US)

(21) Appl. No.: 16/725,673

(22) Filed: Dec. 23, 2019

Related U.S. Application Data
(63) Continuation of application No. 14/752,602, filed on Jun. 26, 2015, now abandoned.
(60) Provisional application No. 62/017,580, filed on Jun. 26, 2014.
(60) Provisional application No. 62/063,870, filed on Oct. 14, 2014.

(51) Int. Cl. C12N 15/10; C12Q 1/6806; C12Q 1/686

(52) U.S. Cl. CPC C12N 15/1058; C12Q 1/686; C12Q 1/6806

(57) ABSTRACT

The present disclosure relates to methods and systems for sample processing and analyzing when the total quantity of input sample is low or when a target of interest is present as a relatively minor or rare population within the overall sample. The disclosure particularly relates to analyzing nucleic acid samples, including samples where a target nucleic acid of interest is present as a relatively low proportion of the overall nucleic acids.

### CROSS-REFERENCE

This application is a continuation of U.S. application Ser. No. 14/752,602, filed Jun. 25, 2015, which claims priority to U.S. Provisional Patent Application No. 62/017,580 filed Jun. 26, 2014 and U.S. Provisional Patent Application No. 62/063,870 filed Oct. 14, 2014 each of which applications is herein incorporated by reference in its entirety for all purposes.

### BACKGROUND

Nucleic acids sequencing is widely used to obtain information in various biomedical contexts, including diagnostics, prognostics, biotechnology, and forensic biology. Sequencing may involve basic methods including Maxam-Gilbert sequencing and chain-termination methods, or de novo sequencing methods including shotgun sequencing and bridge PCR, or next-generation methods including polony sequencing, 454 pyrosequencing, Illumina sequencing, SOLiD sequencing, Ion Torrent semiconductor sequencing, HeliScope single molecule sequencing, SMRT® sequencing, and others. Most sequencing applications require a minimum amount of sample input, which normally varies from hundreds of nanograms to tens of micrograms. Such a requirement for a relatively high input of starting material may cause a significant impediment to numerous applications, particularly applications where a minimal amount of starting material is available. Examples of such applications include non-invasive prenatal diagnosis (NIPD), where only a small minority of DNA is of fetal origin, and cancer diagnosis where often the vast majority of a sample is made up of normal healthy cells and only a tiny amount originated from tumor or cancer cells. There is a need in the art to develop methods and compositions for nucleic acid sequencing of samples with starting quantities of sample nucleic acids that are relatively small, or where the nucleic acids of interest in a sample make up a relatively small proportion of the overall nucleic acids present. The present disclosure addresses these and a variety of other needs.

### SUMMARY

This disclosure provides methods and systems for analyzing nucleic acids, particularly where the input nucleic acid quantity is low. In one aspect, the disclosure provides a method of analyzing nucleic acids that includes providing a collection of nucleic acids derived from a nucleic acid sample, where the collection of nucleic acids includes nucleic acid molecules at an amount of less than 50 nanograms (ng); amplifying the collection of nucleic acids within partitions to form amplification products of the collection of nucleic acids; pooling the collection of nucleic acids and the amplification products to form a pooled mixture; and detecting nucleic acid sequences of at least a portion of nucleic acids within the pooled mixture.

In some embodiments, after providing the collection of nucleic acids and prior to the amplifying, the method includes combining the collection of nucleic acids with a plurality of oligonucleotides releasably connected to beads to form a mixture, partitioning the mixture into a the partitions, and releasing the oligonucleotides from the beads within the partitions. In some embodiments, each of the plurality of oligonucleotides comprises at least a constant region and a variable region. In some embodiments, the constant region comprises a barcode sequence. In some embodiments, the barcode sequence is between about 6 nucleotides and about 20 nucleotides in length. In some embodiments, the variable region comprises a primer sequence. In some embodiments, the oligonucleotides function as primers in the amplifying of the collection of nucleic acids. In some embodiments, the oligonucleotides are released from the beads upon exposure to one or more stimuli (e.g., pH, light, chemical species and/or reducing agent (e.g., dithiothreitol (DTT) or tris(2-carboxylethyl)phosphine (TCEP)).

In some embodiments, the detecting is completed at an accuracy greater than 90%. In some embodiments, the detecting is completed at an accuracy greater than 95%. In some embodiments, the detecting is completed at an accuracy greater than 99%. In some embodiments, the detecting comprises detecting at least 90% of the nucleic acids within the collection of nucleic acids. In some embodiments, the detecting comprises detecting sequences of a minor population within the collection of nucleic acids, which minor population makes up less than 50% of the collection of nucleic acids. In some embodiments, the minor population makes up less than 25% of the collection of nucleic acids. In some embodiments, the minor population makes up less than 10% of the collection of nucleic acids. In some embodiments, the minor population makes up less than 5% of the collection of nucleic acids.

In some embodiments, the amount is less than 40 ng. In some embodiments, the amount is less than 20 ng. In some embodiments, the amount is less than 10 ng. In some embodiments, the amount is less than 5 ng. In some embodiments, the amount is less than 1 ng. In some embodiments, the amount is less than 0.1 ng.

In some embodiments, the partitions comprise droplets (e.g., fluid droplets, such as aqueous droplets within a water-in-oil emulsion), microcapsules, wells or tubes. In some embodiments, the partitions are generated by a microfluidic device.

In some embodiments, the collection of nucleic acids is derived from a bodily fluid such as, for example, a bodily fluid comprising blood, plasma, serum, or urine. In some embodiments, at least a subset of the collection of nucleic acids is derived from one or more circulating tumor cells (e.g., such as one or more circulating tumor cells obtained from a non-conserved sample or from a formaldehyde fixed and paraffin embedded sample) and/or a tumor. In some embodiments, the collection of nucleic acids is derived from a tissue biopsy. In some embodiments, the collection of nucleic acids comprises fetal nucleic acids. In some embodiments, less than 5% of nucleic acids of the collection of nucleic acids comprises fetal nucleic acids. In some embodiments, the nucleic acid sample comprises a cellular sample. In some embodiments, the cellular sample comprises less than 5% circulating tumor cells. In some embodiments, the cellular sample comprises less than 5% tumor cells.

In some embodiments, the nucleic acid sample is derived from a live sample, a non-conserved sample, a preserved sample, an embalmed sample and/or a fixed sample. In some embodiments, the sample is an embedded sample. In some embodiments, the sample is a formaldehyde fixed and paraffin embedded sample.

In another aspect, the disclosure provides a method of analyzing nucleic acids that includes amplifying a collection of nucleic acids derived from a nucleic acid sample within partitions to form amplification products of the collection of nucleic acids; pooling the collection of nucleic acids and the amplification products to form a pooled mixture; and detecting nucleic acid sequences of a minor population within the collection of nucleic acids in the pooled mixture, where the minor population makes up less than 50% of the collection of nucleic acids.

In some embodiments, the method includes, prior to amplifying the collection of nucleic acids, combining the collection of nucleic acids with a plurality of oligonucleotides releasably connected to beads to form a mixture, partitioning the mixture into the partitions, and releasing the oligonucleotides from the beads within the partitions. In some embodiments, each of the plurality of oligonucleotides comprises at least a constant region and a variable region. In some embodiments, the constant region comprises a barcode sequence. In some embodiments, the variable region comprises a primer sequence. In some embodiments, the oligonucleotides function as primers in amplifying the collection of nucleic acids. In some embodiments, the oligonucleotides are released from the beads upon exposure to one or more stimuli (e.g., pH, light, chemical species and/or reducing agent).

In some embodiments, the minor population makes up less than 40%. In some embodiments, the minor population makes up less than 30%. In some embodiments, the minor population makes up less than 20%. In some embodiments, the minor population makes up less than 10%. In some embodiments, the minor population makes up less than 5%. In some embodiments, the minor population makes up less than 1%. In some embodiments, the minor population makes up less than 0.1%. In some embodiments, the minor population comprises tumor nucleic acids. In some embodiments, the minor population comprises fetal nucleic acids. In some embodiments, the minor population comprises circulating tumor cell nucleic acids.

In some embodiments, the partitions comprise droplets, microcapsules, wells or tubes. In some embodiments, the partitions are generated by a microfluidic device. In some embodiments, the collection of nucleic acids is derived from a bodily fluid such as, for example, a bodily fluid that comprises blood, plasma, serum, or urine. In some embodiments, the collection of nucleic acids is derived from a tissue biopsy.

In another aspect, the disclosure provides a method of analyzing nucleic acids that includes providing a collection of nucleic acids derived from a nucleic acid sample, where the collection of nucleic acids includes nucleic acid molecules at an amount of less than 50 nanograms (ng); combining the collection of nucleic acids with a plurality of oligonucleotides to form a mixture, where each of the oligonucleotides comprises at least a constant region and a variable region, which constant region comprises a barcode sequence; partitioning the mixture into a plurality of partitions and amplifying the collection of nucleic acids within the partitions to form amplification products of the collection of nucleic acids; pooling the collection of nucleic acids and the amplification products to form a pooled mixture; and detecting nucleic acid sequences of at least a portion of nucleic acids within the pooled mixture at a sensitivity of at least 90%.

In some embodiments, the collection of nucleic acids includes nucleic acid molecules at an amount of less than 40 ng. In some embodiments, the collection of nucleic acids includes nucleic acid molecules at an amount of less than 20 ng. In some embodiments, the collection of nucleic acids includes nucleic acid molecules at an amount of less than 10 ng. In some embodiments, the collection of nucleic acids includes nucleic acid molecules at an amount of less than 5 ng. In some embodiments, the collection of nucleic acids includes nucleic acid molecules at an amount of less than 1 ng. In some embodiments, the collection of nucleic acids includes nucleic acid molecules at an amount of less than 0.1 ng.

In some embodiments, the variable region comprises a primer sequence. In some embodiments, the oligonucleotides function as primers in amplifying the collection of nucleic acids. In some embodiments, the detecting includes detecting nucleic acid sequences of at least a portion of nucleic acids within the pooled mixture at a sensitivity of at least 95%. In some embodiments, the detecting includes detecting nucleic acid sequences of at least a portion of nucleic acids within the pooled mixture at a sensitivity of at least 99%.

In another aspect, the disclosure provides a method for analyzing a nucleic acid sequence that includes providing partitions comprising nucleic acid molecules generated from a nucleic acid sample; pooling the nucleic acid molecules from the partitions into a nucleic acid mixture; subjecting the nucleic acid mixture to nucleic acid sequencing to generate sequencing reads comprising nucleic acid sequences of the nucleic acid molecules; using a programmed computer processor to analyze the sequencing reads and identify at least one contaminant read in the sequencing reads that is associated with a contaminant nucleic acid molecule in the nucleic acid mixture; removing the contaminant read from the sequencing reads; and generating a sequence of the nucleic acid sample from the sequencing reads with the contaminant read removed.

In some embodiments, amount of the contaminant nucleic acid molecule in the nucleic acid mixture is less than 50%, less than 20%, less than 10%, less than 5%, less than 1%, less than 0.1%, less than 0.01%, less than 0.001% or less than 0.0001% of the nucleic acid molecules in the nucleic acid mixture.

In some embodiments, the at least one contaminant read comprises a plurality of contaminant reads that are associated with contaminant nucleic acid molecules. In some embodiments, the sequence is generated at an accuracy of at least 90%, at least 95% or at least 99%. In some embodiments, the partitions comprise fluid droplets, such as, for example, aqueous droplets within a water-in-oil emulsion.

In some embodiments, the contaminant read is identified by determining sequence overlap(s) among subsets of the sequencing reads and identifying the contaminant read if overlap(s) for a given one of the sequencing reads is less than 50% with respect to all of the subsets, less than 25% with respect to all of the subsets, less than 10% with respect to all of the subsets, less than 5% with respect to all of the subsets, less than 1% with respect to all of the subsets or less than 0.1% with respect to all of the subsets. In some embodiments, the contaminant read is identified by determining sequence overlap(s) among subsets of the sequencing reads and identifying the contaminant read if the sequence of the given one of the sequence reads does not overlap with respect to all of the subsets.

In some embodiments, the contaminant read is identified by comparing the sequencing reads to a reference, and identifying a given sequencing read of the sequencing reads as the contaminant read if the given sequencing read overlaps with the reference at less than 50%, at less than 25%, at less than 10%, at less than 5%, at less than 1% or at less than 0.1%. In some embodiments, the contaminant read is identified by comparing the sequencing reads to a reference and identifying the given sequencing read of the sequencing reads as the contaminant read if the given sequencing does not overlap with the reference.

In some embodiments, the contaminant read is identified by comparing the sequencing reads to one another to identify sequence overlap(s) among the sequencing reads, and identifying a given one of the sequencing reads as the contaminant read if its sequence overlap with other sequencing reads among the sequencing reads is less than 50%, is less than 25%, is less than 10%, is less than 5%, is less than 1% or is less than 0.1%. In some embodiments, the contaminant read is identified by comparing the sequencing reads to one another to identify sequence overlap(s) among the sequencing reads and identifying the given one of the sequencing reads as the contaminant read if its sequence does not overlap with a sequence of the other sequencing reads among the sequencing reads.

In some embodiments, providing partitions comprising nucleic acid molecules generated from the nucleic acid sample includes generating barcoded fragments or copies thereof corresponding to each of the nucleic acid molecules in the partitions. In some embodiments, the sequencing reads comprise barcoded fragment reads comprising nucleic acid sequences of the barcoded fragments or copies thereof. In some embodiments, the contaminant read is identified by identifying a given one of the barcoded fragment reads as the contaminant read if sequence regions to which the given barcoded fragment read maps map barcoded fragment reads having common barcode sequences between the sequence regions of less than 20%, less than 15%, less than 10%, less than 5%, less than 3% or less than 0.1% of the total barcoded fragment reads mappable to the sequence regions.

In some embodiments, the contaminant read is identified by mapping the sequence reads to their sequence region(s) and identifying a given sequence read of the sequence reads as the contaminant read if, when mapped to its sequence region(s), the given sequence read overlaps with less than 10, less than 5, less than 3 or less than 1 or no other reads of the sequence reads when mapped to their sequence region(s).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a flow diagram for example processing a sample for sequencing.

FIG. 2 schematically illustrates an example microfluidic channel structure for co-partitioning samples and beads.

FIG. 3 schematically illustrates an example process for amplification and barcoding of samples.

FIG. 4 provides a schematic illustration of an example of the use of barcoding of sequences in attributing sequence data to their origins.

FIG. 5 provides a schematic illustration of an example computer control system.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. GENERAL OVERVIEW

This disclosure provides methods and systems useful in sample processing and analysis when the starting material is of relatively low quantity or when a target of interest makes up only a small percentage of the total starting material. The methods and systems provided herein are particularly useful for nucleic acid sequencing applications in which the starting nucleic acids (e.g., DNA, mRNA, etc.)—or starting target nucleic acids—are present in small quantities, or where nucleic acids that are targeted for analysis, are present at a relatively low proportion of the total nucleic acids within a sample. The methods and systems provided herein generally involve partitioning the starting sample material into discrete, segregated units; applying an identifying bar-code to the material in the discrete units so that material can be identified on a unit-by-unit basis; pooling the material from the units; sequencing the pooled material; and analyzing the sequencing information in order to detect or quantify nucleic acids of interest.

The described methods and systems provide significant advantages over current nucleic acid sequencing technologies and their associated sample preparation methods. For example, the methods and systems are particularly useful in being able to characterize nucleic acids where the total amount of input nucleic acids is very low. In many nucleic acid analysis systems, a critical limitation lies in the systems' inabilities to analyze very small amounts of nucleic acids. This creates difficulties when analyzing rare events, individual cells, or difficult to obtain or difficult to process samples. By way of example, many current state of the art sequencing systems require starting quantities of nucleic acids for analysis in the range of from 50-100 nanograms (ng) for Illumina sequencing systems, to 500 ng of starting nucleic acids for Pacific Biosciences SMRT sequencing, all the way up to 1 microgram (μg) for Ion Torrent sequencing systems.

In addition to be valuable in the analysis and characterization of nucleic acids where the amount of input nucleic acids is low, the methods and systems described herein also provide significant benefits when analyzing samples for nucleic acids that are present as a low proportion of overall nucleic acids in the sample being analyzed, both when the amount of sample nucleic acids is at an absolute low level, e.g., as described above, and where it is present at a low relative proportion. By way of example, most sequencing technologies rely upon the broad amplification of target nucleic acids in a sample in order to create enough material for the sequencing process. These amplification processes can cause a loss of information, particularly when the sample is a heterogeneous population that contains a minor population of interest, e.g., where a target nucleic acid of interest is present as a relatively low proportion (e.g., less than 20%) of the overall nucleic acids. In particular, broad amplification of the nucleic acids within a sample can preferentially amplify the major population, and overwhelm the signal from minor populations of a sample. The major populations of nucleic acids within a sample may, in some cases, outcompete minor populations during the amplification process such that the major populations are preferentially amplified. An example of a sample with major and minor nucleic acid populations is a tissue biopsy sample that may primarily contain healthy tissue and very little diseased tissue such as tissue from a tumor. Only a small percentage of nucleic acids (e.g., DNA) extracted from such a sample may thus represent the diseased or abnormal population (e.g., less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, less than 0.001% etc.). A typical amplification method such as PCR may quickly amplify the DNA from the healthy tissue to the detriment of amplification, and even the exclusion of amplification of the DNA from the tumor cells. Such amplification results from several factors, including, e.g., the progress of geometric amplification, where a sample starting from a higher quantity quickly outpaces amplification of the minority component. It can also result from resource utilization, in which the more rapidly-growing population quickly commands the available resources for amplification, e.g., primers, polymerases and nucleotides, to amplify that majority component to the exclusion of amplification of the minority component. Furthermore, because these amplification reactions are typically carried out in a pooled context, the origin of an amplified sequence, in terms of the specific chromosome, polynucleotide or organism may not be preserved during the process.

In certain aspects, the methods and systems provided herein partition individual or small numbers of nucleic acids so that they are allocated into separate reaction volumes, e.g., in droplets or other partitions, in which those nucleic acid components may be initially amplified. During this initial amplification, a unique barcode is coupled to the components that are in those separate reaction volumes. Separate, partitioned amplification of the different components, as well as application of a unique barcode sequence, allows for the preservation of the contributions of each sample component, as well as attribution of its origin, through the sequencing process, including subsequent amplification processes, e.g., PCR or other amplification processes. Methods of partitioning samples and bar-coding are described in detail in U.S. patent application Ser. No. 14/316,383 filed Jun. 26, 2014, as well as U.S. Provisional Patent Application Nos. 61/940,318, filed Feb. 7, 2014 and 61/991,018, filed May 9, 2014, the full disclosures of which are hereby incorporated herein by reference in their entireties for all purposes.

The methods and systems disclosed herein are useful in a wide-range of settings. For example, the methods and systems can be used for clinical diagnostics, particularly to diagnose, or differentially diagnose, cancers including solid organ cancers and blood cancers or to detect fetal aneuploidy in samples obtained from pregnant women. The methods and systems can also be used for biological research, particularly biomedical research. The methods and systems can also be used to characterize populations of organisms (e.g., such as a microbiome), as well as in forensics and environmental testing.

### II. WORK FLOW OVERVIEW

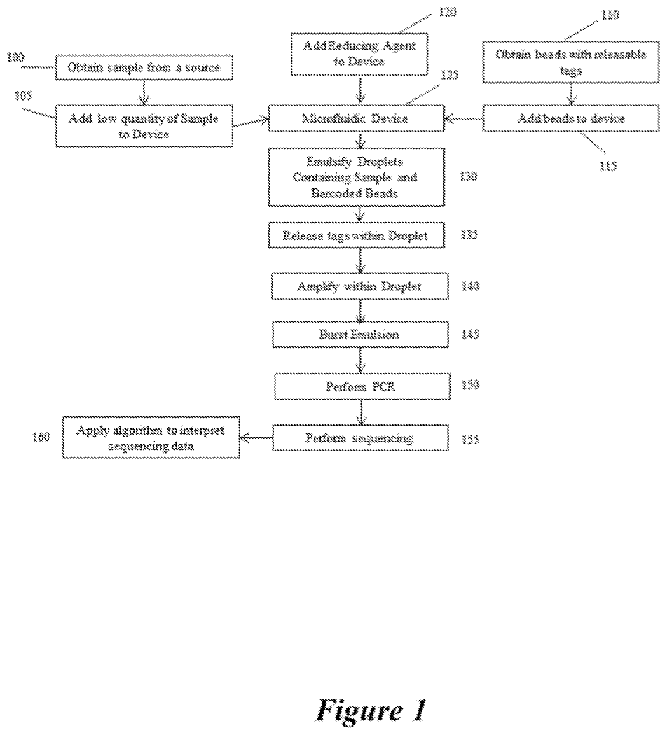

FIG. 1 illustrates an example method for barcoding and subsequently sequencing a sample nucleic acid, particularly where the sample is of relatively-low quantity or where a target population is a relatively minor population within the sample (e.g., less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, less than 0.001% etc.). First, a sample comprising nucleic acid may be obtained from a source, 100, and a set of barcoded beads may also be obtained, 110. The beads can be linked to oligonucleotides containing one or more barcode sequences, as well as a primer, such as a random N-mer or other primer. In some cases, the barcode sequences are releasable from the barcoded beads, e.g., through cleavage of a linkage between the barcode and the bead or through degradation of the underlying bead to release the barcode, or a combination of the two. For example, in some cases, the barcoded beads can be degraded or dissolved by an agent, such as a reducing agent to release the barcode sequences. In this example, a low quantity of the sample comprising nucleic acid, 105, barcoded beads, 115, and, in some cases, other reagents, e.g., a reducing agent, 120, are combined and subject to partitioning. By way of example, such partitioning may involve introducing the components to a droplet generation system, such as a microfluidic device, 125. With the aid of the microfluidic device 125, a water-in-oil emulsion 130 may be formed, wherein the emulsion contains aqueous droplets that contain sample nucleic acid, 105, reducing agent, 120, and barcoded beads, 115. The reducing agent may dissolve or degrade the barcoded beads, thereby releasing the oligonucleotides with the barcodes and random N-mers from the beads within the droplets, 135. The random N-mers may then prime different regions of the sample nucleic acid, resulting in amplified copies of the sample after amplification, wherein each copy is tagged with a barcode sequence, 140. In some cases, each droplet contains a set of oligonucleotides that contain identical barcode sequences and different random N-mer sequences. Subsequently, the emulsion is broken, 145 and additional sequences (e.g., sequences that aid in particular sequencing methods, additional barcodes, etc.) may be added, via, for example, amplification methods, 150 (e.g., PCR). Sequencing may then be performed, 155, and an algorithm applied to interpret the sequencing data, 160. Sequencing algorithms are generally capable, for example, of performing analysis of barcodes to align sequencing reads and/or identify the sample from which a particular sequence read belongs.

Described herein are methods and systems for characterizing nucleic acids with low input quantity. As used herein and as described below, low input quantity of nucleic acids generally refers to a low aggregate quantity of sample nucleic acids introduced into a work flow. In some embodiments, the term refers to the aggregate quantity of sample nucleic acids introduced into a device such as a microfluidic device. As described further herein, the quantity of nucleic acids may be expressed in terms of mass or genomic equivalents, e.g., the number of genomic equivalents introduced into the workflow, for example when analyzing whole genomic samples. As will be appreciated, this can vary from the mass-based input quantity numbers described above, depending upon the size of the genome of the organism being analyzed. Input sample nucleic acids also encompasses the total amount of sample nucleic acids that is introduced, regardless of the state (e.g., intact, fragmented, extracted, extracted and fragmented, fragmented and size-selected, etc.).

In one exemplary aspect, the methods and systems described in the disclosure provide for depositing or partitioning individual or small amounts of samples (e.g., nucleic acids) into discrete partitions, where each partition maintains separation of its own content from the contents in other partitions. As used herein, the partitions refer to containers or vessels that may include a variety of different forms, e.g., wells, tubes, micro or nanowells, through holes, or the like. In some aspects, however, the partitions are flowable within fluid streams. These vessels may be comprised of, e.g., microcapsules or micro-vesicles that have an outer barrier surrounding an inner fluid center or core, or they may be a porous matrix that is capable of entraining and/or retaining materials within its matrix. In some aspects, however, these partitions may comprise droplets of aqueous fluid within a non-aqueous continuous phase, e.g., an oil phase. A variety of different vessels are described in, for example, U.S. patent application Ser. No. 13/966,150, filed Aug. 13, 2013. Likewise, emulsion systems for creating stable droplets in non-aqueous or oil continuous phases are described in detail in, e.g., U.S. Patent Publication No. 2010/0105112, the full disclosure of which is entirely incorporated herein by reference.

In the case of droplets in an emulsion, partitioning of sample materials, e.g., nucleic acids, into discrete partitions may generally be accomplished by flowing an aqueous, sample containing stream, into a junction into which is also flowing a non-aqueous stream of partitioning fluid, e.g., a fluorinated oil, such that aqueous droplets are created within the flowing stream partitioning fluid, where such droplets include the sample materials. As described below, such droplets also typically include co-partitioned barcode oligonucleotides. The relative amount of sample materials within any particular partition may be adjusted by controlling a variety of different parameters of the system, including, for example, the concentration of sample in the aqueous stream, the flow rate of the aqueous stream and/or the non-aqueous stream, and the like. The partitions described herein are often characterized by having extremely small volumes. For example, in the case of droplet based partitions, the droplets may have overall volumes that are less than 1000 pL, less than 900 pL, less than 800 pL, less than 700 pL, less than 600 pL, less than 500 pL, less than 400 pL, less than 300 pL, less than 200 pL, less than 100 pL, less than 50 pL, less than 20 pL, less than 10 pL, or even less than 1 pL. Where co-partitioned with beads, it will be appreciated that the sample fluid volume within the partitions may be less than 90% of the above described volumes, less than 80%, less than 70%, less than 60%, less than 50%, less than 40%, less than 30%, less than 20%, or even less than 10% the above described volumes. In some cases, the use of low reaction volume partitions is particularly advantageous in performing reactions with very small amounts of starting reagents, e.g., input nucleic acids.

Once the samples are introduced into their respective partitions, in accordance with the methods and systems described herein, the contents within partitions are generally provided with unique identifiers such that, upon characterization of those contents they may be attributed as having been derived from their respective origins. Accordingly, the samples are typically co-partitioned with the unique identifiers (e.g., barcode sequences). In some aspects, the unique identifiers are provided in the form of oligonucleotides that comprise nucleic acid barcode sequences that may be attached to those samples. The oligonucleotides are partitioned such that as between oligonucleotides in a given partition, the nucleic acid barcode sequences contained therein are the same, but as between different partitions, the oligonucleotides can have differing barcode sequences. In some aspects, only one nucleic acid barcode sequence will be associated with a given partition, although in some cases, two or more different barcode sequences may be present.

The nucleic acid barcode sequences can include from 6 to about 20 or more nucleotides within the sequence of the oligonucleotides. These nucleotides may be completely contiguous, i.e., in a single stretch of adjacent nucleotides, or they may be separated into two or more separate subsequences that are separated by one or more nucleotides. Typically, separated subsequences may typically be from about 4 to about 16 nucleotides in length.

The co-partitioned oligonucleotides also typically comprise other functional sequences useful in the processing of the nucleic acids from the co-partitioned cells. These sequences include, e.g., targeted or random/universal amplification primer sequences for amplifying the genomic DNA from the individual cells within the partitions while attaching the associated barcode sequences, sequencing primers, hybridization or probing sequences, e.g., for identification of presence of the sequences, or for pulling down barcoded nucleic acids, or any of a number of other potential functional sequences. Again, co-partitioning of oligonucleotides and associated barcodes and other functional sequences, along with sample materials is described in, for example, U.S. patent Ser. No. Application Nos. 61/940,318, filed Feb. 7, 2014, 61/991,018, Filed May 9, 2014, and U.S. patent application Ser. No. 14/316,383 filed Jun. 26, 2014, previously incorporated by reference.

Briefly, in one exemplary process, beads are provided that each may include large numbers of the above described oligonucleotides releasably attached to the beads, where all of the oligonucleotides attached to a particular bead may include the same nucleic acid barcode sequence, but where a large number of diverse barcode sequences may be represented across the population of beads used. Typically, the population of beads may provide a diverse barcode sequence library that may include at least 1000 different barcode sequences, at least 10,000 different barcode sequences, at least 100,000 different barcode sequences, or in some cases, at least 1,000,000 different barcode sequences. Additionally, each bead may typically be provided with large numbers of oligonucleotide molecules attached. In particular, the number of molecules of oligonucleotides including the barcode sequence on an individual bead may be at least bout 10,000 oligonucleotides, at least 100,000 oligonucleotide molecules, at least 1,000,000 oligonucleotide molecules, at least 100,000,000 oligonucleotide molecules, and in some cases at least 1 billion oligonucleotide molecules.

The oligonucleotides may be releasable from the beads upon the application of a particular stimulus to the beads. In some cases, the stimulus may be a photo-stimulus, e.g., through cleavage of a photo-labile linkage that may release the oligonucleotides. In some cases, a thermal stimulus may be used, where elevation of the temperature of the beads environment may result in cleavage of a linkage or other release of the oligonucleotides form the beads. In some cases, a chemical stimulus may be used that cleaves a linkage of the oligonucleotides to the beads, or otherwise may result in release of the oligonucleotides from the beads.

In accordance with the methods and systems described herein, the beads including the attached oligonucleotides may be co-partitioned with the individual samples, such that a single bead and a single sample are contained within an individual partition. In some cases, where single bead partitions are desired, the relative flow rates of the fluids can be controlled such that, on average, the partitions contain less than one bead per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions.

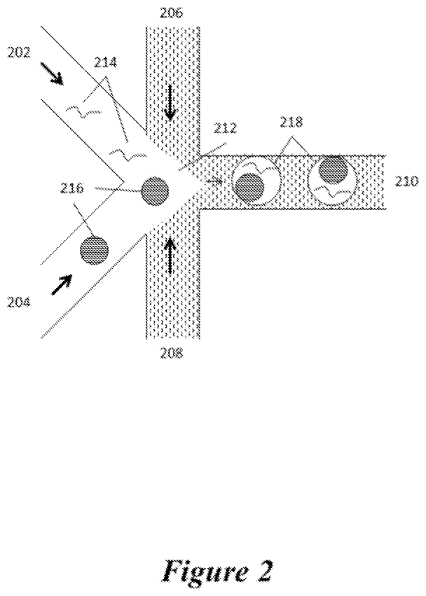

As noted above, while single bead occupancy may be a desired state, it will be appreciated that multiply occupied partitions, or unoccupied partitions may often be present. An example of a microfluidic channel structure for co-partitioning samples and beads comprising barcode oligonucleotides is schematically illustrated in FIG. 2. As shown, channel segments 202, 204, 206, 208 and 210 are provided in fluid communication at channel junction 212. An aqueous stream comprising the individual samples 214 is flowed through channel segment 202 toward channel junction 212. As described elsewhere herein, these samples may be suspended within an aqueous fluid prior to the partitioning process.

Concurrently, an aqueous stream comprising the barcode carrying beads 216 is flowed through channel segment 204 toward channel junction 212. A non-aqueous partitioning fluid is introduced into channel junction 212 from each of side channels 206 and 208, and the combined streams are flowed into outlet channel 210. Within channel junction 212, the two combined aqueous streams from channel segments 202 and 204 are combined, and partitioned into droplets 218, that include co-partitioned samples 214 and beads 216. As noted previously, by controlling the flow characteristics of each of the fluids combining at channel junction 212, as well as controlling the geometry of the channel junction, one can optimize the combination and partitioning to achieve a desired occupancy level of beads, samples or both, within the partitions 218 that are generated.

As will be appreciated, a number of other reagents may be co-partitioned along with the samples and beads, including, for example, chemical stimuli, nucleic acid extension, transcription, and/or amplification reagents such as polymerases, reverse transcriptases, nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

Once co-partitioned, the oligonucleotides disposed upon the bead may be used to barcode and amplify the partitioned samples. A particularly elegant process for use of these barcode oligonucleotides in amplifying and barcoding samples is described in detail in U.S. Patent Application Nos. 61/940,318, filed Feb. 7, 2014, 61/991,018, Filed May 9, 2014, and U.S. patent application Ser. No. 14/316,383 filed Jun. 26, 2014, previously incorporated by reference. Briefly, in one aspect, the oligonucleotides present on the beads that are co-partitioned with the samples and released from their beads into the partition with the samples. The oligonucleotides typically include, along with the barcode sequence, a primer sequence at its 5′ end. This primer sequence may be a random oligonucleotide sequence intended to randomly prime numerous different regions of the samples, or it may be a specific primer sequence targeted to prime upstream of a specific targeted region of the sample.

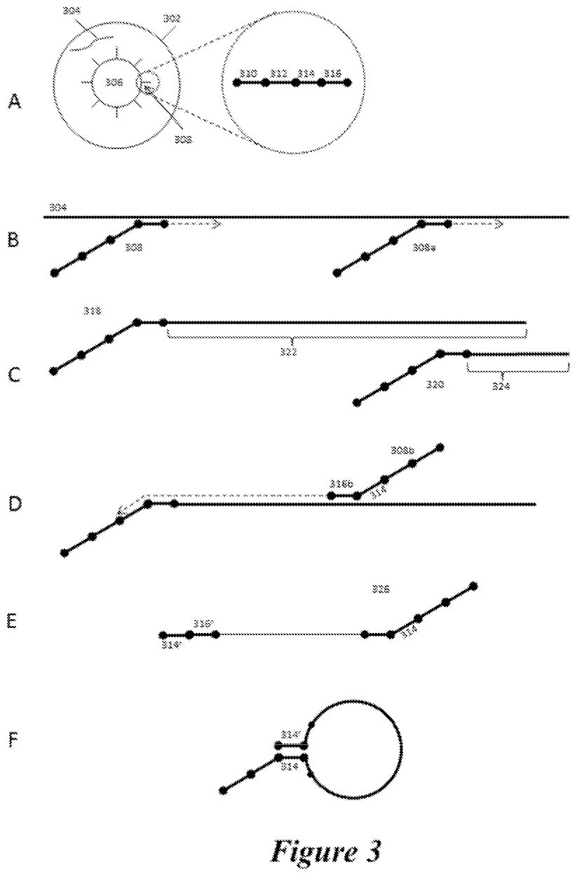

Once released, the primer portion of the oligonucleotide can anneal to a complementary region of the sample. Extension reaction reagents, e.g., DNA polymerase, nucleoside triphosphates, co-factors (e.g., Mg′ or Mn′ etc.), that are also co-partitioned with the samples and beads, then extend the primer sequence using the sample as a template, to produce a complementary fragment to the strand of the template to which the primer annealed, with complementary fragment includes the oligonucleotide and its associated barcode sequence. Annealing and extension of multiple primers to different portions of the sample may result in a large pool of overlapping complementary fragments of the sample, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow the formation of a hairpin structure or partial hairpin structure, the reduces the ability of the molecule to be the basis for producing further iterative copies. A schematic illustration of one example of this is shown in FIG. 3.

As the figure shows, oligonucleotides that include a barcode sequence are co-partitioned in, e.g., a droplet 302 in an emulsion, along with a sample nucleic acid 304. As noted elsewhere herein, the oligonucleotides 308 may be provided on a bead 306 that is co-partitioned with the sample nucleic acid 304, which oligonucleotides can be releasable from the bead 306, as shown in panel A. The oligonucleotides 308 include a barcode sequence 312, in addition to one or more functional sequences, e.g., sequences 310, 314 and 316. For example, oligonucleotide 308 is shown as comprising barcode sequence 312, as well as sequence 310 that may function as an attachment or immobilization sequence for a given sequencing system, e.g., a P5 sequence used for attachment in flow cells of an Illumina Hiseq or Miseq system. As shown, the oligonucleotides also include a primer sequence 316, which may include a random or targeted N-mer for priming replication of portions of the sample nucleic acid 304. Also included within oligonucleotide 308 is a sequence 314 which may provide a sequencing priming region, such as a “read1” or R1 priming region, that is used to prime polymerase mediated, template directed sequencing by synthesis reactions in sequencing systems. In some cases, the barcode sequence 312, immobilization sequence 310 and R1 sequence 314 may be common to all of the oligonucleotides attached to a given bead. The primer sequence 316 may vary for random N-mer primers, or may be common to the oligonucleotides on a given bead for certain targeted applications.

Based upon the presence of primer sequence 316, the oligonucleotides are able to prime the sample nucleic acid as shown in panel B, which allows for extension of the oligonucleotides 308 and 308a using polymerase enzymes and other extension reagents also co-portioned with the bead 306 and sample nucleic acid 304. As shown in panel C, following extension of the oligonucleotides that, for random N-mer primers, would anneal to multiple different regions of the sample nucleic acid 304; multiple overlapping complements or fragments of the nucleic acid are created, e.g., fragments 318 and 320. Although including sequence portions that are complementary to portions of sample nucleic acid, e.g., sequences 322 and 324, these constructs are generally referred to herein as comprising fragments of the sample nucleic acid 304, having the attached barcode sequences.

The barcoded nucleic acid fragments may then be subjected to characterization, e.g., through sequence analysis, or they may be further amplified in the process, as shown in panel D. For example, additional oligonucleotides, e.g., oligonucleotide 308b, also released from bead 306, may prime the fragments 318 and 320. In particular, again, based upon the presence of the random N-mer primer 316b in oligonucleotide 308b (which in some cases may be different from other random N-mers in a given partition, e.g., primer sequence 316), the oligonucleotide anneals with the fragment 318, and is extended to create a complement 326 to at least a portion of fragment 318 which includes sequence 328, that comprises a duplicate of a portion of the sample nucleic acid sequence. Extension of the oligonucleotide 308b continues until it has replicated through the oligonucleotide portion 308 of fragment 318. As noted elsewhere herein, and as illustrated in panel D, the oligonucleotides may be configured to prompt a stop in the replication by the polymerase at a desired point, e.g., after replicating through sequences 316 and 314 of oligonucleotide 308 that is included within fragment 318. As described herein, this may be accomplished by different methods, including, for example, the incorporation of different nucleotides and/or nucleotide analogues that are not capable of being processed by the polymerase enzyme used. For example, this may include the inclusion of uracil containing nucleotides within the sequence region 312 to prevent a non-uracil tolerant polymerase to cease replication of that region. As a result a fragment 326 is created that includes the full-length oligonucleotide 308b at one end, including the barcode sequence 312, the attachment sequence 310, the R1 primer region 314, and the random N-mer sequence 316b. At the other end of the sequence can be included the complement 316′ to the random N-mer of the first oligonucleotide 308, as well as a complement to all or a portion of the R1 sequence, shown as sequence 314′. The R1 sequence 314 and its complement 314′ are then able to hybridize together to form a partial hairpin structure 328. As will be appreciated because the random N-mers differ among different oligonucleotides, these sequences and their complements would not be expected to participate in hairpin formation, e.g., sequence 316′, which is the complement to random N-mer 316, would not be expected to be complementary to random N-mer sequence 316b. This would not be the case for other applications, e.g., targeted primers, where the N-mers would be common among oligonucleotides within a given partition.

By forming these partial hairpin structures, it allows for the removal of first level duplicates of the sample sequence from further replication, e.g., preventing iterative copying of copies. The partial hairpin structure also provides a useful structure for subsequent processing of the created fragments, e.g., fragment 326.

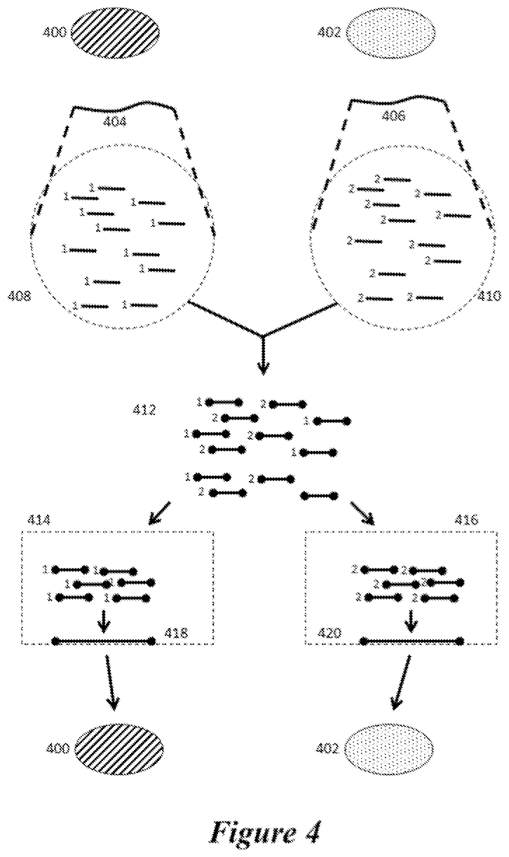

All of the fragments from multiple different partitions may then be pooled for sequencing on high throughput sequencers as described herein. Because each fragment is coded as to its partition of origin, the sequence of that fragment may be attributed back to its origin based upon the presence of the barcode. This is schematically illustrated in FIG. 4. As shown in one example, a nucleic acid 404 originated from a first source 400 (e.g., normal cells), and a nucleic acid 406 derived from a differing source 402 (e.g., tumor cells) are each partitioned along with their own sets of barcode oligonucleotides as described above. In some instances normal cells, tumor cells or both are obtained from a tissue or fluid comprising cells (i.e. from a “sample”) selected from the group consisting of live sample, a non-conserved sample, preserved sample, embalmed sample, embedded sample, fixed sample, or any combination thereof. In some examples, the tissue or cell is both embedded and either preserved, embalmed or fixed. In some instances the sample is both embedded and fixed. In some examples normal cells, tumor cells or both are formaldehyde (e.g. formalin) fixed and paraffin embedded (FFPE).

Within each partition, each nucleic acid 404 and 406 is then processed to separately provide overlapping set of second fragments of the first fragment(s), e.g., second fragment sets 408 and 410. This processing also provides the second fragments with a barcode sequence that is the same for each of the second fragments derived from a particular first fragment. As shown, the barcode sequence for second fragment set 408 is denoted by “1” while the barcode sequence for fragment set 410 is denoted by “2”. A diverse library of barcodes may be used to differentially barcode large numbers of different fragment sets. However, it is not necessary for every second fragment set from a different first fragment to be barcoded with different barcode sequences. In some cases, multiple different first fragments may be processed concurrently to include the same barcode sequence. Diverse barcode libraries are described in detail elsewhere herein.

The barcoded fragments, e.g., from fragment sets 408 and 410, may then be pooled for sequencing using, for example, sequence by synthesis technologies available from Illumina or Ion Torrent division of Thermo Fisher, Inc. Once sequenced, the sequence reads 412 can be attributed to their respective fragment set, e.g., as shown in aggregated reads 414 and 416, at least in part based upon the included barcodes, and, in some cases, in part based upon the sequence of the fragment itself. The attributed sequence reads for each fragment set are then assembled to provide the assembled sequence for each sample fragment, e.g., sequences 418 and 420, which in turn, may be further attributed back to their respective origins, e.g., normal cells 400 and tumor cells 402. Methods for genomic assembly are described in, e.g., U.S. Provisional Patent Application No. 62/017,589 filed on Jun. 26, 2014, the full disclosure of which is hereby incorporated by reference in its entirety. In some instances normal cells, tumor cells or both are obtained from a tissue or cell-sample (i.e. sample) selected from the group consisting of live sample, a non-conserved sample, preserved sample, embalmed sample, embedded sample, fixed sample, or any combination thereof. In some examples, the tissue or cell is both embedded and either preserved, embalmed or fixed. In some instances the tissue or cell is both embedded and fixed. In some examples normal cells, tumor cells or both are formaldehyde (e.g. formalin) fixed and paraffin embedded (FFPE) tissue.

Embedding is the process in which a tissue or a cell is placed into molds along with liquid embedding material (e.g. gel, resin, wax, or any combination thereof) which may subsequently be hardened. Embedding may be achieved through a cooling process (e.g. when at least one paraffin wax is used as an embedding medium). Embedding may be achieved through a heating (e.g. curing) process (e.g. when at least one epoxy resin is used as an embedding medium). Embedding may use acrylic resins, which may be polymerized though the use of heat, ultraviolet light, or chemical catalysts. Embedding can be done by using frozen, tissue in an aqueous medium. Pre-frozen tissues may be placed into molds with liquid embedding material (e.g. a water-based glycol, cryogel, or resin) which may then be frozen to form hardened blocks. In some instances, the embedding process utilizes resin(s). In some instances, the embedding process utilizes wax. The wax may be animal wax, plant wax, petroleum wax, synthetic wax or any combination thereof. The animal wax may be tallow, beeswax, spermaceti or lanolin. The plant wax may be epicuticular, coticular wax, or any combination thereof. The plant wax can be carnauba wax, candelilla wax, ouricury wax, soy wax, or a combination thereof. The wax may be petroleum derived wax such as paraffin. A paraffin wax may be comprised of n-alkane having a carbon chain length of at least 10, 15, 20, 25, 30, 35, 40, 45 or 50 carbon atoms and at most 15, 20, 25, 30, 35, 40, 45, 50 or 55 carbon atoms, or any combination of the aforementioned n-alkanes. In some examples, a resin is any component of a liquid that sets into a hard lacquer or enamel-like finish. Resins may comprise natural resins such as amber, kauri gum, rosin, copal, dammar, mastic, sandarac, frankincense, elemi, turpentine, copaiba, ammoniacum, asafoetida, gamboge, myrrh, or scammony. The resin may be derived from a wooden source (e.g., a tree, such as, for example, a pine tree). The resin may be a synthetic resin such as nail polish, epoxy resins, thermosetting plastic, or any combination thereof. Gel may be any dilute cross-linked molecular array, which exhibits no flow when in the steady-state. Gels may be hydrogels, xerogels or hydrogels. Gels may be naturally produced, synthetic or any combination thereof. Gels may comprise agarose, methylcellulose, hyaluronan, caragreenan, gelatin, or any combination thereof.

Fixation is the process that preserves biological tissue or a cell from decay, thereby preventing autolysis or putrefaction. In some examples, a fixed tissue or fixed cell is one that is preserved from decay. Decay may involve decomposition (i.e. rotting), which is the process by which organic substances are broken down into simpler forms of matter. The preservation from decay may prevent autolysis, putrefaction or both. A fixed tissue may preserve its cells, its tissue components or both. Tissue fixation may be done through a crosslinking fixative by forming covalent bonds between proteins in the tissue or cell to be fixed. Fixation may anchor soluble proteins to the cytoskeleton of a cell. Fixation may form a rigid cell, a rigid tissue or both. Fixation may be achieved through use of chemicals such as formaldehyde (e.g. formalin), gluteraldehyde, ethanol, methanol, acetic acid, osmium tetraoxide, potassium dichromate, chromic acid, potassium permanganate, Zenker's fixative, picrates, Hepes-glutamic acid buffer-mediated organic solvent protection effect (HOPE), or any combination thereof. Formaldehyde may be used as a mixture of about 37% formaldehyde gas in aqueous solution on a weight by weight basis. The aqueous formaldehyde solution may additionally comprise about 10-15% of an alcohol (e.g. methanol), forming a solution termed “formalin.” A fixative-strength (10%) solution would equate to a 3.7% solution of formaldehyde gas in water. Formaldehyde may be used as at least 5%, 8%, 10%, 12% or 15% Neutral Buffered Formalin (NBF) solution (i.e. fixative strength). Formaldehyde may be used as 3.7% to 4.0% formaldehyde in phosphate buffered saline (i.e. formalin). In some instances, fixation is performed using at least 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11.0, 11.5, 12.0, 12.5, 13.0, 13.5, 14.0, 14.5, or 15.0 percent (%) or more formalin flush or immersion. In some instances, fixation is performed using about 10% formalin flush. Fixative volume can be 10, 15, 20, 25 or 30 times that of tissue on a weight per volume. Subsequent to fixation in formaldehyde, the tissue or cell may be submerged in alcohol for long term storage. In some cases, the alcohol is methanol, ethanol, propanol, butanol, an alcohol containing five or more carbon atoms, or any combination thereof. The alcohol may be linear or branched. The alcohol may be at least 50%, 60%, 70%, 80% or 90% alcohol in aqueous solution. In some examples, the alcohol is 70% ethanol in aqueous solution.

Embalming preserves a tissue or a cell from natural decomposition. An embalmed sample may be a sanitized sample, presentable sample or preserved sample. A presentable sample is an in vitro sample that preserves its appearance in its former in vivo state. In some embodiments, an embalmed tissue or embalmed cell is a tissue that was immersed in an embalming fluid, or a tissue to which the embalming fluid was injected to. The embalming fluid may at least temporarily delay decomposition and restore a natural appearance. The embalming fluid comprises preservatives, sanitizers, disinfectants, or any combination thereof. The embalming fluid may comprise formaldehyde, glutaraldehyde, ethanol, humectants, or a combination thereof. The formaldehyde content in an embalming fluid may ranges from 5 to 35 percent (%); the alcohol content in an embalming fluid may range from 9 to 56 percent (%). The alcohol may be any of the aforementioned alcohols or any combination thereof. In some examples, the alcohol is ethanol.

A preserved sample is one in which decomposition is delayed as compared to the natural sample (i.e. without the addition of preservatives). Decomposition may occur as a consequence of microbial growth, undesirable chemical changes, or both. A preserved tissue or cell may be a tissue or a cell that is contacted with nitrates, ammonia, benzoic acid, sodium benzoate, hydrobenzoate, lactic acid, propionic acid, sulfur dioxide, sulfites, sorbic acid, ascorbic acid, butylated hydroxytoluene, butylated hydroxyanisole, gallic acid, tocopherol(s), disodium EDTA, citric acid, tartaric acid, lecithin, phenolase, castor oil, alcohol, hops, rosemary, diatomaceous earth, or any combination thereof.

In some examples, the sample may be both embedded and either embalmed, preserved or fixed. For example, the sample can be both fixed and embedded. Fixation may be achieved using any of the aforementioned fixation materials or methods delineated. Embedding may be achieved using any of the aforementioned embedding materials or methods delineated. For instance, the sample may be both formaldehyde fixed and paraffin embedded. In some instances fixative for paraffin embedded tissues uses neutral buffered formalin (NBF). NBF may be equivalent to 4% paraformaldehyde in a buffered solution. In some instances NBF further includes a preservative (e.g. an alcohol). The alcohol may be any of the aforementioned alcohols. Fixation, may take at least 12, 25, 36, 48, or 60 hours. Fixation, may take at most 25, 36, 48, 60 or 72 hours. The fixation may be conducted at room temperature. Paraffin embedding may comprise tissue dehydration. The tissue dehydration may be accomplished through a series of graded alcohol baths to displace the water, subsequently infiltrated by wax. The infiltrated tissues may then be embedded into wax. The alcohol may be ethanol. The wax may be any of the abovementioned waxes. In some instances, the wax is a paraffin wax. The paraffin wax may be a solid at room temperature having a melting point of at least about 45, 50, 55, 60, 65, 70, 75 or 80 degrees Celsius (° C.). The paraffin wax may be a solid at room temperature having a melting point of at most about 45, 50, 55, 60, 65, 70, 75 or 80 degrees Celsius (° C.). In some instances, the paraffin wax has a melting point of from at least 56° C. to at most 58° C. Formalin-fixed, paraffin-embedded (FFPE) tissues can be stored for a prolonged time of at least 5, 10, 15, 50, 75, 100, 150, 200, 250, 500, 1000 years or more. The storing for a prolonged time may be at room temperature. Formalin-fixed, paraffin-embedded (FFPE) tissues can be stored indefinitely at room temperature. In some instances, nucleic acids (e.g., DNA, RNA or both) may be recovered from the FFPE tissue after fixation.

### III. SAMPLES

a. Types of Samples

The methods and systems of this disclosure may be used with any suitable sample that can be introduced into a microfluidic device and partitioned into discrete compartments. Exemplary samples may include polynucleotides, nucleic acids, oligonucleotides, circulating cell-free nucleic acid, circulating tumor nucleic acid (e.g., circulating tumor DNA), circulating tumor cell (CTC) nucleic acids, nucleic acid fragments, nucleotides, DNA, RNA, peptide polynucleotides, complementary DNA (cDNA), double stranded DNA (dsDNA), single stranded DNA (ssDNA), plasmid DNA, cosmid DNA, chromosomal DNA, genomic DNA (gDNA), viral DNA, bacterial DNA, mitochondrial DNA (mtDNA), cell-free DNA, cell free fetal DNA (cffDNA), ribosomal DNA (rDNA), messenger RNA (mRNA), ribosomal RNA (rRNA), transfer RNA (tRNA), nRNA, siRNA, snRNA, snoRNA, scaRNA, microRNA, single-stranded RNA (ssRNA), dsRNA, viral RNA, cRNA, and the like. In some cases, the samples may contain proteins or polypeptides.

The sample may comprise any combination of any nucleotides. The nucleotides may be naturally occurring or synthetic. In some cases, the nucleotides may be oxidized or methylated. The nucleotides may include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate.

The sample may be any synthetic nucleic acid, such as peptide nucleic acid (PNA), analog nucleic acid, glycerol nucleic acid (GNA), threose nucleic acid (TNA), locked nucleic acid (LNA) or other synthetic polymers with nucleotide side chains.

The sample may have different degrees of purity. In some cases, the sample may be a DNA sample wherein more than 5%, 10%, 15%, 20%, 30%, 40%, 50%, 60%, 70%, 80%, 90%, 95%, 96%, 97%, 98%, 99%, 99.1%, 99.2%, 99.5%, or 99.9% of the sample is made up of DNA. In some cases, the sample may be a DNA sample wherein less than 0.1%, 0.2%, 0.3%, 0.5%, 1%, 2%, 3%, 4%, 5%, 10%, 15%, 20%, 30%, 40%, 50%, 60%, 70%, 80%, 90%, 95%, 96%, 97%, 98%, 99%, 99.1%, 99.2%, 99.5%, or 99.9% of the sample is made up of DNA. In some cases, the sample may be a RNA sample wherein more than 5%, 10%, 15%, 20%, 30%, 40%, 50%, 60%, 70%, 80%, 90%, 95%, 96%, 97%, 98%, 99%, 99.1%, 99.2%, 99.5%, or 99.9% of the sample is made up of RNA. In some cases, the sample may be a RNA sample wherein less than 0.1%, 0.2%, 0.3%, 0.5%, 1%, 2%, 3%, 4%, 5%, 10%, 15%, 20%, 30%, 40%, 50%, 60%, 70%, 80%, 90%, 95%, 96%, 97%, 98%, 99%, 99.1%, 99.2%, 99.5%, or 99.9% of the sample is made up of RNA. In some cases the sample is 100% DNA; in some cases the sample is 100% RNA.

The sample may contain a mixture of different species. In some cases, the sample contains a mixture of DNA, RNA, protein, and lipid, or any combination thereof, or any relative ratio thereof. For example, the sample may contain DNA, RNA, and protein in the following ratio: 1:1:50. In another example, the sample may contain a mixture of different types of DNA (e.g., a mixture of synthetic and naturally-occurring DNA; a mixture of maternal and fetal DNA; etc.). In yet another example, a sample may contain a mixture of different types of RNA (e.g., a mixture containing mRNA, tRNA and/or rRNA). Samples may also be present within cells that are disposed within the partitions, e.g., as described in U.S. Patent Application No. 62/017,558 filed Jun. 26, 2014, previously incorporated by reference.

b. Source of Samples

Any substance that comprises nucleic acid may be the source of a sample. The substance may be a fluid, e.g., a biological fluid. A fluidic substance may include, but not limited to, blood, cord blood, saliva, urine, sweat, serum, semen, vaginal fluid, gastric and digestive fluid, spinal fluid, placental fluid, cavity fluid, ocular fluid, serum, breast milk, lymphatic fluid, or combinations thereof.

The substance may be from solid tissue, for example, a biological tissue or collection of cells or biopsy. The substance may comprise normal healthy tissues. The tissues may be associated with various types of organs. Non-limiting examples of organs may include brain, liver, lung, kidney, prostate, ovary, spleen, lymph node (including tonsil), thyroid, pancreas, heart, skeletal muscle, intestine, larynx, esophagus, stomach, or combinations thereof.

The substance may comprise tumors. Tumors may be benign (non-cancer) or malignant (cancer). Non-limiting examples of tumors may include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastrointestinal system carcinomas, colon carcinoma, pancreatic cancer, breast cancer, genitourinary system carcinomas, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, endocrine system carcinomas, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, or combinations thereof. The tumors may be associated with various types of organs. Non-limiting examples of organs may include brain, liver, lung, kidney, prostate, ovary, spleen, lymph node (including tonsil), thyroid, pancreas, heart, skeletal muscle, intestine, larynx, esophagus, stomach, or combinations thereof.

The substance may comprise a mix of normal healthy tissues or tumor tissues. The tissues may be associated with various types of organs. Non-limiting examples of organs may include brain, liver, lung, kidney, prostate, ovary, spleen, lymph node (including tonsil), thyroid, pancreas, heart, skeletal muscle, intestine, larynx, esophagus, stomach, or combinations thereof.

In some cases, the substance comprise a variety of cells, including but not limited to: eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, etc. In some cases, the substance may comprise contents of a cell, such as, for example, the contents of a single cell or the contents of multiple cells.

In some cases the cells are normal cells, tumor cells or both and are obtained from a tissue sample or cell-sample (i.e. sample) selected from the group consisting of live sample, a non-conserved sample, preserved sample, embalmed sample, embedded sample, fixed sample, or any combination thereof. In some examples, the tissue sample or cell sample is both embedded and either preserved, embalmed or fixed. In some instances the tissue sample or cell sample is both embedded and fixed. In some examples tissue sample, cell sample or both are formaldehyde (e.g. formalin) fixed and paraffin embedded (FFPE).

Samples may be obtained from various subjects. A subject may be a living subject or a dead subject. In some cases, the subject is a mammalian subject, such as, for example, a human subject. Examples of subjects may include, but not limited to, humans, mammals, non-human mammals, rodents, amphibians, reptiles, canines, felines, bovines, equines, goats, ovines, hens, avines, mice, rabbits, insects, slugs, microbes, bacteria, parasites, or fish. In some cases the subject is healthy, such as a healthy man, woman, child, or infant. In some cases, the subject may be a patient who has, is suspected of having, or at a risk of developing a disease or disorder. In some cases, the subject may be a pregnant woman. In some case, the subject may be a normal healthy pregnant woman. In some cases, the subject may be a pregnant woman who is at a risking of carrying a baby with certain birth defects.

A sample may be obtained from a subject by various approaches. For example, a sample may be obtained from a subject through accessing the circulatory system (e.g., intravenously or intra-arterially via a syringe or other apparatus), collecting a secreted biological sample (e.g., saliva, sputum urine, feces, etc.), surgically (e.g., biopsy) acquiring a biological sample (e.g., intra-operative samples, post-surgical samples, etc.), swabbing (e.g., buccal swab, oropharyngeal swab), or pipetting, or by any other means for obtaining tissue fluid or other samples from subjects.

### IV. QUANTITY OF INPUT SAMPLES

a. Total Input of Samples

The quantity of total input sample (e.g., DNA, RNA, etc.) that can be used in the methods provided herein may vary. The methods and systems provided herein are particularly useful for when the input sample is of low quantity; but they may also be used with high quantities of input samples. In some cases, the quantity of input samples may be about 1 fg, 5 fg, 10 fg, 25 fg, 50 fg, 100 fg, 200 fg, 300 fg, 400 fg, 500 fg, 600 fg, 700 fg, 800 fg, 900 fg, 1 pg, 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 2 μg, 3 μg, 4 μg, 5 μg, 6 μg, 7 μg, 8 μg, 9 μg, 10 μg, 15 μg, or 20 μg. In some cases, the quantity of input samples may be at least about 1 fg, 5 fg, 10 fg, 25 fg, 50 fg, 100 fg, 200 fg, 300 fg, 400 fg, 500 fg, 600 fg, 700 fg, 800 fg, 900 fg, 1 pg, 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 2 μg, 3 μg, 4 μg, 5 μg, 6 μg, 7 μg, 8 μg, 9 μg, 10 μg, 15 μg, 20 μg, or more. In some cases, the quantity of input samples may be no more or may be less than about 20 μg, 15 μg, 10 μg, 9 μg, 8 μg, 7 μg, 6 μg, 5 μg, 4 μg, 3 μg, 2 μg, 900 ng, 800 ng, 700 ng, 600 ng, 500 ng, 400 ng, 300 ng, 200 ng, 100 ng, 90 ng, 80 ng, 75 ng, 70 ng, 65 ng, 60 ng, 59 ng, 58 ng, 57 ng, 56 ng, 55 ng, 54 ng, 53 ng, 52 ng, 51 ng, 50 ng, 49 ng, 48 ng, 47 ng, 46 ng, 45 ng, 44 ng, 43 ng, 42 ng, 41 ng, 40 ng, 35 ng, 30 ng, 25 ng, 20 ng, 15 ng, 10 ng, 5 ng, 2.5 ng, 1 ng, 900 pg, 800 pg, 700 pg, 600 pg, 500 pg, 400 pg, 300 pg, 200 pg, 100 pg, 50 pg, 25 pg, 10 pg, 5 pg, 1 pg, 900 fg, 800 fg, 700 fg, 600 fg, 500 fg, 400 fg, 300 fg, 200 fg, 100 fg, 50 fg, 25 fg, 10 fg, 5 fg, 1 fg or less. In some cases, the quantity of input sample may fall within a range between any two of the values described herein.

In some cases, about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents of nucleic acids may be used as an input sample. In some cases, less than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents of nucleic acids may be used. In some cases, more than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents of nucleic acids may be used. In some cases, the number of genome equivalents of nucleic acids used may fall within a range between any two of the values described herein.

In some cases, the input samples may constitute about 1×, 2×, 5×, 10×, 15×, 20×, 30×, 40×, or 50× coverage of the of the underlying larger genetic component (e.g., genome). In some cases, the input samples may constitute less than about 1×, 2×, 5×, 10×, 15×, 20×, 30×, 40×, or 50× coverage of the of the underlying larger genetic component. In some cases, the input samples may constitute greater than about 1×, 2×, 5×, 10×, 15×, 20×, 30×, 40×, or 50× coverage of the of the underlying larger genetic component. In some cases, the input samples may cover the underlying larger genetic component at a range between any two of the values described herein.

b. Input Quantity of Target Components within a Sample

In some examples, input sample may comprise various types of components (e.g., nucleic acids), or components originated from differing sources. The target components or the components of interest (e.g., components associated with a disease or disorder) within a certain sample may make up a certain percentage of the total input. For example, a sample may be comprised of mostly normal tissue DNA (e.g., 95% or more, 99% or more) and very little (e.g., 5% or less, 1% or less) tumor or cancer cell DNA with the latter one being the one of interest. The methods and systems provided herein are particularly useful when a target component (e.g., nucleic acid) makes up only a minor proportion of the overall sample. For example, the methods and systems are particularly useful to detect rare populations of nucleic acids (e.g., cell-free nucleic acids, cell-free fetal nucleic acids, cell-free fetal nucleic acids, cell-free nucleic acids originating from tumors, etc.) or nucleic acids derived from rare populations of cells. In some cases, the target components may make up a high percentage of the total input. In some cases, the target components may make up a low percentage of the total input. In some cases, the target components may make up about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 91%, 92%, 93%, 94%, 95%, 96%, 97%, 98%, 99%, or 99.9% of the total input. In some cases, the target components may make up at least about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 91%, 92%, 93%, 94%, 95%, 96%, 97%, 98%, 99%, or 99.9% of the total input. In some cases, the target components may make up less than about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 91%, 92%, 93%, 94%, 95%, 96%, 97%, 98%, 99%, or 99.9% of the total input. In some cases, the target components may make up a range of percentages falling into any two of the values described herein.

In some embodiments, the sample may comprise nucleic acids obtained from a body fluid, particularly blood or urine. The sample may comprise circulating cell-free nucleic acids and/or nucleic acids associated with circulating tumor cells. The cells may be obtained from a tissue selected from the group consisting of live tissue, non-conserved tissue, preserved tissue, embalmed tissue, embedded tissue, fixed tissue, or any combination thereof. In some examples, the cells are both embedded and either preserved, embalmed or fixed. In some instances the cells are both embedded and fixed. In some examples the cells are formaldehyde (e.g. formalin) fixed and paraffin embedded (FFPE).

In some cases, a target population of interest (e.g., cell-free nucleic acids, fetal nucleic acids, nucleic acids associated with circulating tumor cells, etc.) may comprise less than 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20% of the total sample input. In some embodiments, the input sample is a cellular sample (e.g., a blood sample) wherein less than 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% of the total number of cells within the sample are made up of cancer cells (e.g., circulating tumor cells). Methods and systems for analyzing cellular samples are described in U.S. Provisional Patent Application No. 62/017,558, filed on Jun. 26, 2014, the full disclosure of which is hereby incorporated by reference for all purposes.

The quantity of input target components may vary. In some cases, about 1 fg, 5 fg, 10 fg, 25 fg, 50 fg, 100 fg, 200 fg, 300 fg, 400 fg, 500 fg, 600 fg, 700 fg, 800 fg, 900 fg, 1 pg, 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 15 μg, or 20 μg of target components may be inputted. In some cases, at least about 1 fg, 5 fg, 10 fg, 25 fg, 50 fg, 100 fg, 200 fg, 300 fg, 400 fg, 500 fg, 600 fg, 700 fg, 800 fg, 900 fg, 1 pg, 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 15 μg, 20 μg or more of target components may be inputted. In some cases, no more than or less than about 20 μg, 15 μg, 10 μg, 9 μg, 8 μg, 7 μg, 6 μg, 5 μg, 4 μg, 3 μg, 2 μg, 1 μg, 900 ng, 800 ng, 700 ng, 600 ng, 500 ng, 400 ng, 300 ng, 200 ng, 100 ng, 90 ng, 80 ng, 75 ng, 70 ng, 65 ng, 60 ng, 59 ng, 58 ng, 57 ng, 56 ng, 55 ng, 54 ng, 53 ng, 52 ng, 51 ng, 50 ng, 49 ng, 48 ng, 47 ng, 46 ng, 45 ng, 44 ng, 43 ng, 42 ng, 41 ng, 40 ng, 35 ng, 30 ng, 25 ng, 20 ng, 15 ng, 10 ng, 5 ng, 2.5 ng, 1 ng, 900 pg, 800 pg, 700 pg, 600 pg, 500 pg, 400 pg, 300 pg, 200 pg, 100 pg, 50 pg, 25 pg, 10 pg, 5 pg, 1 pg, 900 fg, 800 fg, 700 fg, 600 fg, 500 fg, 400 fg, 300 fg, 200 fg, 100 fg, 50 fg, 25 fg, 10 fg, 5 fg, 1 fg or less of target components may be inputted. In some cases, the quantity of inputted target components may fall into a range between any of the two values described herein.

In some cases, the input quantity of target components may be about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the input quantity of target components may be less than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the input quantity of target components may be more than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the number of genome equivalents of nucleic acids contained in target components may be falling into a range between any two of the values described herein.

In some cases, the inputted target components may constitute about 1×, 2×, 5×, 10×, 15×, 20×, 30×, 40×, or 50× coverage of the of the underlying larger genetic component (e.g., genome). In some cases, the inputted target components may constitute less than about 1×, 2×, 5×, 10×, 15×, 20×, 30×, 40×, or 50× coverage of the of the underlying larger genetic component. In some cases, the inputted target components may constitute greater than about 1×, 2×, 5×, 10×, 15×, 20×, 30×, 40×, or 50× coverage of the of the underlying larger genetic component. In some cases, the inputted target components may cover the underlying larger genetic component at a range between any two of the values described herein.

c. Input Quantity of Target Sample within a Sample Mixture

In some examples, inputted samples may be a mix of samples originated from varying subjects or sources where target samples may constitute certain percentage of the total input. For example, biological samples for forensic analysis may comprise nucleic acids from differing subjects (e.g., victims, perpetrators, witnesses, crime lab analysts, etc.), while only a portion of the mixture is the target. In some cases, the target sample may constitute a high percentage of the total input. In some cases, the target sample may constitute a low percentage of the total input. In some cases, the target sample may constitute about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 99%, or 99.99% of the total input. In some cases, the target sample may constitute at least about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 99%, or 99.99% of the total input. In some cases, the target sample may constitute no more than or less than about 0.000001%, 0.000005%, 0.0000075%, 0.00001%, 0.00005%, 0.000075%, 0.0001%, 0.0005%, 0.00075%, 0.001%, 0.005%, 0.0075%, 0.01%, 0.05%, 0.075%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 99% or 99.99% of the total input. In some cases, the target sample may constitute a range of percentages falling between any of the two values described herein.

The quantity of target sample included may vary. In some cases, a high quantity of target sample may be included. In some cases, a low quantity of target sample may be included. In some cases, about 1 femtogram (fg), 5 fg, 10 fg, 25 fg, 50 fg, 100 fg, 200 fg, 300 fg, 400 fg, 500 fg, 600 fg, 700 fg, 800 fg, 900 fg, 1 picogram (pg), 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 1 microgram (μg), 2 μg, 3 μg, 4 μg, 5 μg, 6 μg, 7 μg, 8 μg, 9 μg, 10 μg, 15 μg, or 20 μg of target sample may be included. In some cases, at least about 1 fg, 5 fg, 10 fg, 25 fg, 50 fg, 100 fg, 200 fg, 300 fg, 400 fg, 500 fg, 600 fg, 700 fg, 800 fg, 900 fg, 1 pg, 5 pg, 10 pg, 25 pg, 50 pg, 100 pg, 200 pg, 300 pg, 400 pg, 500 pg, 600 pg, 700 pg, 800 pg, 900 pg, 1 ng, 2.5 ng, 5 ng, 10 ng, 15 ng, 20 ng, 25 ng, 30 ng, 35 ng, 40 ng, 41 ng, 42 ng, 43 ng, 44 ng, 45 ng, 46 ng, 47 ng, 48 ng, 49 ng, 50 ng, 51 ng, 52 ng, 53 ng, 54 ng, 55 ng, 56 ng, 57 ng, 58 ng, 59 ng, 60 ng, 65 ng, 70 ng, 75 ng, 80 ng, 90 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 15 μg, 20 μg or more of target sample may be included. In some cases, no more than or less than about 20 μg, 15 μg, 10 μg, 9 μg, 8 μg, 7 μg, 6 μg, 5 μg, 4 μg, 3 μg, 2 μg, 1 μg, 900 ng, 800 ng, 700 ng, 600 ng, 500 ng, 400 ng, 300 ng, 200 ng, 100 ng, 90 ng, 80 ng, 75 ng, 70 ng, 65 ng, 60 ng, 59 ng, 58 ng, 57 ng, 56 ng, 55 ng, 54 ng, 53 ng, 52 ng, 51 ng, 50 ng, 49 ng, 48 ng, 47 ng, 46 ng, 45 ng, 44 ng, 43 ng, 42 ng, 41 ng, 40 ng, 35 ng, 30 ng, 25 ng, 20 ng, 15 ng, 10 ng, 5 ng, 2.5 ng, 1 ng, 900 pg, 800 pg, 700 pg, 600 pg, 500 pg, 400 pg, 300 pg, 200 pg, 100 pg, 50 pg, 25 pg, 10 pg, 5 pg, 1 pg, 900 fg, 800 fg, 700 fg, 600 fg, 500 fg, 400 fg, 300 fg, 200 fg, 100 fg, 50 fg, 25 fg, 10 fg, 5 fg, 1 fg or less of target sample may be included. In some cases, the quantity of target sample may fall into a range between any two of the values described herein.

In some cases, the input quantity of target sample may be about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the input quantity of target sample may be less than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the input quantity of target sample may be more than about 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, 20000, 25000, 30000, 35000, 40000, 45000, or 50000 genome equivalents. In some cases, the input quantity of target sample may be between any two of the numbers described herein.

In some cases, the target sample included may constitute about 1×, 2×, 5×, 10×, 15×, 20×, 30×, 40×, or 50× coverage of the of the underlying larger genetic component (e.g., genome). In some cases, the target sample included may constitute less than about 1×, 2×, 5×, 10×, 15×, 20×, 30×, 40×, or 50× coverage of the of the underlying larger genetic component. In some cases, the target sample included may constitute greater than about 1×, 2×, 5×, 10×, 15×, 20×, 30×, 40×, or 50× coverage of the of the underlying larger genetic component. In some cases, the target sample included may cover the underlying larger genetic component at a range between any two of the values described herein.

d. Samples in Partitions

Partitioning of samples may be carried out so as to provide a desired level of sample nucleic acids into the partitions in order to achieve the goals of the analysis. For example, it can be desired that sample nucleic acids are partitioned so as to minimize the probability that any duplicate nucleic acid portions (e.g., target nucleic acids) from the sample are present within a single partition. This may generally be achieved by providing the sample nucleic acids within the aqueous stream that is being partitioned, at a sufficiently low concentration, or limiting dilution, so that only a certain amount of nucleic acid is partitioned within any single partition. Typically, sample nucleic acids may be treated as to provide sample nucleic acid fragments that include fragments that are from about 10 kilobases (kb) to about 100 kb in length, or from about 10 kb to about 30 kb in length. In such cases, it can be generally desirable to ensure that nucleic acids within a partition comprise from about 100 to about 500 fragments. In other applications, it may be desirable to provide nucleic acids within a partition at widely varied amounts, including down to as low as a single nucleic acid fragment within a partition, all the way up to providing a whole genome, or entire contents of a cell, within a single partition.

In the context of some aspects of the systems and methods described herein, in some cases, it can be desired to control the number of beads that are co-partitioned with the sample nucleic acids. In some cases, it can be desired to provide partitions which have only a single bead disposed therein, i.e., are “singly occupied”. As alluded to above, this is generally accomplished by controlling one or more of the flow rates of the various fluids that are converging within a droplet generation junction, controlling the dimensions and structure of that junction, and controlling the geometries of the overall channels within the system or device in which the droplets are being generated.

In certain examples, the beads may be partitioned so that a certain percentage of partitions contain no more than one bead. In some cases, about 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one bead. In some cases, at least about 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one bead. In some cases, no more than 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one bead. In some cases, the percentages of partitions that contain no more than one bead may be falling into a range between any two of the values described herein.

In certain examples, a sample is a nucleic acid sample comprising a target nucleic acid (or target nucleic acid population) and may be partitioned so that a certain percentage of partitions contain no more than one target nucleic acid, no more than two target nucleic acids, no more than three target nucleic acids, no more than four target nucleic acids, or no more than five target nucleic acids. In some cases, about 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one target nucleic acid. In some cases, at least about 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one target nucleic acid. In some cases, no more than 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of partitions may contain no more than one target nucleic acid. In some cases, the percentages of partitions that contain no more than one target nucleic acid may fall into a range between any two of the values described herein. In some cases, the partitions comprise on average less than one target nucleic acid, on average less than two target nucleic acids, on average less than three target nucleic acids, on average less than four target nucleic acids, or on average less than five target nucleic acids.

Additionally or alternatively, in some cases, it can be desirable to avoid the creation of excessive number of empty partitions, e.g. partitions that include no beads. As described elsewhere herein in the disclosure, the flow of the fluids directed into the partitioning zone, e.g., sample fluids, bead containing fluid, and/or partitioning fluid may be controlled such that no more than 90%, no more than 80%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 2.5%, or no more than 1% of the generated partitions are unoccupied, i.e., have no beads disposed therein. In most cases, the above noted ranges of unoccupied partitions may be achieved while still providing any of the above-described single occupancy rates. For example, in some cases, the use of the systems and methods of the present disclosure creates resulting partitions that have multiple occupancy rates of from less than 25%, less than 20%, less than 15%, less than 10%, and in some cases, less than 5%, while having unoccupied partitions of from less than 50%, less than 40%, less than 30%, less than 20%, less than 10%, and in some cases, less than 5%.

Although described in terms of providing substantially singly occupied partitions, above, in certain cases, it can be desirable to provide multiply occupied partitions, e.g., containing two, three, four or more beads within a single partition. Likewise, sample quantities with in the partitions may also be adjusted as desired to achieve varied goals. Accordingly, as noted above, the flow characteristics of the sample and/or bead containing fluids and partitioning fluids may be controlled to provide for such multiply occupied partitions or varied sample concentrations or amounts within such partitions. In particular, the flow parameters may be controlled to provide an occupancy rate at greater than 50% of the partitions, greater than 75%, and in some cases greater than 80%, 90%, 95%, or higher.

A number of approaches may be used to generate the partitions as described herein, including bulk partitioning methods, e.g., bulk emulsion forming systems, large scale droplet formation systems, e.g., as provided by Nanomi, Inc., or microfluidic partitioning systems. In some aspects, partitioning systems used herein include those described in U.S. Provisional Patent Application No. 61/977,804, filed Apr. 10, 2014, the full disclosure of which is hereby incorporated by reference in its entirety.

### V. INTRODUCING SAMPLE TO A DEVICE

In any of the various aspects of the present disclosure, a sample obtained from a subject may be introduced into a device or system where the sample can be furthered combined or mixed with other reagents (e.g., functional beads, barcoded beads, reagents necessary for sample amplification, reducing agents, primers, functional sequences, etc.). Devices or systems may include microfluidic devices that include microscale channel networks integrated within a unified body structure, or they may comprise an aggregation of components that provides the fluidics used in the processing of samples. As described herein, the term device is used to describe any configuration of the fluidic functionalities described herein, including the foregoing. The device may or may not comprise a sample loading channel. In some cases, the device may comprise a plurality of sample loading channels. The device may or may not comprise a sample receiving vessel. In some case, the device may comprise one or more of sample receiving vessels. Sample receiving vessels may be permanently associated with the device. Sample receiving vessels may be attached to the device. Sample receiving vessels may be separable with the device. A sample receiving vessel may be of varied shape, size, weight, material and configuration. For examples, a sample receiving vessel may be regularly shaped or irregularly shaped, may be round or oval tubular shaped, may be rectangular, square, diamond, circular, elliptical, or triangular shaped. A sample receiving vessel can be made of any type of materials such as glass, plastics, polymers, metals etc. Non-limiting examples of types of a sample receiving vessel may include a tube, a well, a capillary tube, a cartridge, a cuvette, a centrifuge tube, or a pipette tip. In some cases, the device may comprise a plurality of identical sample receiving vessels. In some cases, the device may comprise a plurality of different sample receiving vessels that may differ in at least one of the factors including size, shape, weight, material and configuration. In some cases, the device may be in communication with one or more other devices (e.g., thermal cycler, sequencer, etc.). In some cases, the device may be part of another device.

In some cases, a sample may be directly introduced or loaded into the device by using certain tools. Non-limiting examples of tools include pipettes, auto-pipettes, electronic pipettes, digital reading pipettes, digital adjustment pipettes, positive displacement pipettes, repeater pipettes, microdispenser pipettes, bottle top dispensers, manual syringes, auto-sampler syringes, analytical electronic syringes, Hamilton syringes, or combinations thereof. In some cases, a sample may be dissolved in, suspended in or mixed with a substance prior to the sample loading. The substance may be a liquid or a gas. The substance may be in communication with one or more of sample loading channels of the device. In some cases, a sample may be introduced to the device by a secondary device, e.g., a syringe pump or a sample dispenser.

A sample may be loaded to the device in a controlled manner. In some cases, the amount of loaded sample may be controlled. In some case, the volume of loaded sample may be controlled. In some cases, the amount of sample loaded may be controlled via the adjustment of the sample-loading rate. In some cases, the volume of sample loaded may be controlled via the adjustment of the sample-loading rate.

One or more types of samples may be introduced into the device. In the case where there is more than one types of samples to be loaded, they may be loaded successively or contemporaneously. In some cases, different types of samples may be loaded via the same loading channel. In some cases, different types of samples may be loaded via various loading channels. In some cases, different types of samples may be loaded into the same sample receiving vessel. In some cases, different types of samples may be loaded into their corresponding sample receiving vessels. In some aspects, a single device or system may include multiple parallel channel or fluidic networks in order to process multiple different samples, while reducing or eliminating potential cross-contamination issues.

A sample may or may not be processed prior to being loaded into the device. In some cases, a sample may be introduced into the device without any further processing. In some cases, a sample may be subjected to one or more processing procedures before being introduced into the device. For example, in the case where a mix of nucleic acids is used as a sample, the mix may be processed such that one or more components within the mix are isolated, extracted or purified before being introduced into the device. For example, in some cases, exomes may be purified from the original nucleic acid sample. In another example, longer sequences of nucleic acids may be fragmented into a variety of smaller sequences prior to the sample loading, which fragments may or may not be subjected to additional processing to enrich for fragments of a desired size or size range, e.g., using a Blue Pippin fragment selection system. In some cases, the sample to be loaded may be pre-mixed with other reagents before being loaded into the device. Non-limiting examples of reagents may include functional beads, barcodes, oligonucleotides, modified nucleotides, native nucleotides, DNA polymerase, RNA polymerase, reverse transcriptase, mutant proofreading polymerase, dTTPs, dUTPs, dCTPs, dGTPs, dATPs, primers, sample index sequences, sequencing primer binding sites, sequencer primer binding sites, reducing agents, or combinations thereof.

Any device as described herein that is capable of receiving the sample and combining the sample with certain reagents for further processing steps may be used. Such a device may be a microfluidic device (e.g., a droplet generator). Examples of such microfluidic devices include those described in detail in U.S. Provisional Patent Application No. 61/977,804, filed Apr. 10, 2014, the full disclosure of which is incorporated herein by reference in its entirety for all purposes.

### VI. PERFORMANCE OF THE TEST

The methods and systems described herein may provide a high accuracy for detecting and analyzing samples with a low input quantity of nucleic acids (e.g., less than 50 nanograms (ng), less than 49 ng, less than 48 ng, less than 47 ng, less than 46 ng, less than 45 ng, less than 44 ng, less than 43 ng, less than 42 ng, less than 41 ng, less than 40 ng, less than 35 ng, less than 30 ng, less than 25 ng, less than 20 ng, less than 15 ng, less than 10 ng, less than 5 ng, less than 2.5 ng, less than 1 ng, less than 0.5 ng, less than 0.1 ng, less than 0.05 ng, less than 0.01 ng, less than 0.005 ng, less than 0.001 ng, etc.). Such accuracy may be at least about 50%, at least about 60%, at least about 70%, at least about 80%, at least about 85%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, at least about 99.9%, at least about 99.99%, at least about 99.999%, or at least about 99.9999%.

Methods and systems described herein may provide a high sensitivity in detecting and analyzing samples with low input quantity of nucleic acids (e.g., less than 50 ng, less than 49 ng, less than 48 ng, less than 47 ng, less than 46 ng, less than 45 ng, less than 44 ng, less than 43 ng, less than 42 ng, less than 41 ng, less than 40 ng, less than 35 ng, less than 30 ng, less than 25 ng, less than 20 ng, less than 15 ng, less than 10 ng, less than 5 ng, less than 2.5 ng, less than 1 ng, less than 0.5 ng, less than 0.1 ng, less than 0.05 ng, less than 0.01 ng, less than 0.005 ng, less than 0.001 ng, etc.). Such sensitivity may be at least about 50%, at least about 60%, at least about 70%, at least about 80%, at least about 85%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, at least about 99.9%, at least about 99.99%, at least about 99.999%, or at least about 99.9999%.

Methods and systems described herein may provide a high specificity in detecting and analyzing samples with low-input quantities of nucleic acids (e.g., less than 50 ng, less than 49 ng, less than 48 ng, less than 47 ng, less than 46 ng, less than 45 ng, less than 44 ng, less than 43 ng, less than 42 ng, less than 41 ng, less than 40 ng, less than 35 ng, less than 30 ng, less than 25 ng, less than 20 ng, less than 15 ng, less than 10 ng, less than 5 ng, less than 2.5 ng, less than 1 ng, less than 0.5 ng, less than 0.1 ng, less than 0.05 ng, less than 0.01 ng, less than 0.005 ng, less than 0.001 ng, etc.). Such specificity may be at least about 50%, at least about 60%, at least about 70%, at least about 80%, at least about 85%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, at least about 99.9%, at least about 99.99%, at least about 99.999%, or at least about 99.9999%.

### VII. APPLICATIONS

a. Diagnosing Cancer and Other Diseases

The methods and systems described herein may be useful in diagnosing cancers or diseases (e.g., dementia) in a subject having, suspected of having, or at risk of having cancers or diseases. In particular, these methods, compositions and systems are useful in detecting cancers by sequencing and characterizing cancer cells.

As described elsewhere herein, cancer cells may be obtained from solid tumors or obtained as circulating tumor cells (collectively “cancer sample”). The solid tumors may be obtained from a live cancer sample, a non-conserved cancer sample, preserved cancer sample, embalmed cancer sample, embedded cancer sample, fixed cancer sample, or any combination thereof. The cancer sample may be both embedded and either preserved, embalmed or fixed. In some instances the cancer sample is both embedded and fixed. In some examples the cancer sample is formaldehyde fixed and paraffin embedded (FFPE).

Analyses of circulating tumor cells (CTCs) are considered as a real-time “liquid biopsy’ in cancer patients and this biopsy may further allow the characterization of specific sub-populations of CTCs, which therefore holds great promise in cancer diagnosis. However, detecting CTCs remains technically challenging as CTCs occur at very low concentrations (1 CTC in the background of millions of normal cells), their identification and characterization require extremely sensitive and specific analytical methods. (Pantel K. et al., Journal of Thoracic Disease, 2012, 4(5): 446-447), the full disclosure of which is hereby incorporated by reference in its entirety.

Most nucleic acid sequencing technologies derive the DNA that they sequence from collections of cells obtained from tissue or other samples. The cells are typically processed, en masse, to extract the genetic material that represents an average of the population of cells, which is then processed into sequencing ready DNA libraries that are configured for a given sequencing technology. Following from this processing, absent a cell specific marker, attribution of genetic material as being contributed by a subset of cells or all cells in a sample is virtually impossible in such an ensemble approach.

In addition to the inability to attribute characteristics to particular subsets of populations of cells, such ensemble sample preparation methods also are, from the outset, predisposed to primarily identifying and characterizing the majority constituents in the sample of cells, and are not designed to be able to pick out the minority constituents, e.g., genetic material contributed by one cell, a few cells, or a small percentage of total cells in the sample.

In contrast, the methods and systems provided herein may partition or allocate individual or small numbers of nucleic acids, e.g., circulating tumor-associated DNA, into separate reaction volumes or partitions (e.g., droplets), in which those nucleic acids or nucleic acid components may be initially amplified by primer sequences (e.g., random N-mers) contained in oligonucleotides that are releasably attached to beads. Furthermore, during this initial amplification process, a unique identifier (e.g., barcode sequences) may be coupled to the sample nucleic acid or nucleic acid components that are in those separate partitions.

As described elsewhere herein, upon the generation of partitions, by adjusting the flow rates of sample stream, bead stream or both, or by altering the geometry of channel junction, partitions with desired sample (or target nucleic acid)/bead occupancy may be created. Separate, partitioned amplification of the different sample or components, along with the application of the unique identifier, allows for the preservation of the contributions of each sample component, as well as attribution to their respective origin (e.g., normal cell, tumor cell, circulating tumor cell, etc.), through a sequencing process. In some cases, additional rounds of amplification processes may be performed.

b. Identifying Fetal Aneuploidy

Aneuploidy is a condition in which the chromosome number is not an exact multiple of the number characteristic of a particular species. An extra or missing chromosome is a common cause of genetic disorders including human birth defects. For example, Down syndrome (DS) (also “trisomy 21” herein) is a genetic disorder caused by the presence of all or part of a third copy of chromosome 21. Edwards syndrome (also “trisomy 18” herein) is a chromosomal disorder caused by the presence of all, or part of, an extra 18th chromosome. Patau syndrome, or trisomy 13, is a syndrome caused by a chromosomal abnormality, in which some or all of the cells of the body contain extra genetic material from chromosome 13. Conventional methods of diagnosing chromosomal abnormities such as chorionic villus sampling and amniocentesis may impose potentially significant risks to both fetus and the mother. Noninvasive screening of fetal aneuploidy using maternal serum markers and ultrasound is available but has very limited reliability. (Fan et al. PNAS, 2008, 105(42): 16266-16271), the full disclosure of which is hereby incorporated by reference in its entirety for all purposes.

Recent discovery of the presence of cell-free fetal nucleic acids in maternal circulation has led to the development of noninvasive prenatal genetic tests for aneuploidies. Cell-free fetal DNA (cffDNA), a fetal DNA circulating freely in the maternal blood stream, originates from the trophoblasts making up the placenta. The fetal DNA is fragmented and makes its way into the maternal bloodstream via shedding of the placental microparticles into the maternal bloodstream. However, measuring aneuploidy through the analysis of cell-free fetal DNA remains challenging because of the high background of maternal DNA. It is estimated that fetal DNA often constitutes less than 10% of total DNA in maternal cell-free plasma.

The methods, compositions and systems described herein are useful in detecting and diagnosing fetal aneuploidies by sequencing and analyzing the cell-free fetal DNA in maternal blood or other body fluids. Methods and systems for detecting copy number variations and phasing of haplotypes are described in U.S. Provisional Application No. 62/017,808, filed Jun. 26, 2014, the full disclosure of which is hereby incorporated by reference in its entirety for all purposes.

In an exemplary process, individual or small number of nucleic acids with differing origins or sources (e.g., cell-free maternal DNA, cell-free fetal DNA, etc.) may be separately partitioned into a plurality of reaction volumes, or partitions (e.g., droplets). Meanwhile, a plurality of beads with releasably attached oligonucleotides may be partitioned into the same separate partitions such that each partition may contain both beads and sample nucleic acids. As described elsewhere herein, the occupancy rates of partitions may be adjusted such that each partition may contain certain numbers of samples and/or oligonucleotide attached beads, through altering the flow rates of sample stream, bead stream or the both, or the geometry of the channel junction. Additionally, the partitioning process may also be controlled such that certain percentages of partitions may include no more than one target sample nucleic acid (e.g., a cell-free fetal DNA). For example, in some cases, the use of systems and methods provided herein may create less than 90%, less than 70%, less than 60%, less than 50%, less than 40%, less than 30%, less than 20%, less than 10%, or less than 5% of the occupied resulting partitions that contain more than one target nucleic acid (e.g. a cell-free fetal DNA). In some cases, the partitioning process may be adjusted such that a substantial percentage of the overall occupied partitions may include at least a target sample and a bead. For example, at least 5%, at least 10%, at least 20%, at least 30%, at least 40%, at least 50%, at least 60%, at least 70%, at least 80%, at least 90%, or at least 99% of the partitions may be so occupied. In some cases, it may be desirable to provide a single target sample and a single bead within a partition and at least 5%, at least 10%, at least 20%, at least 30%, at least 40%, at least 50%, at least 60%, at least 70%, at least 80%, at least 90%, or at least 99% of the partitions may be so occupied.

After generating the partitions, the oligonucleotides associated to a given bead may be released into the partition and attach to one or more target samples within a given partition. The common barcode sequences and random N-mers included in oligonucleotides may be used to identify the origin of the sample sequence and prime multiple fragments of the sample sequence within each given partition, during an initial amplification process. These initially amplified fragments of the samples may then be pooled and sequenced (e.g., using any suitable sequencing method, including those described elsewhere herein). The identities of the barcodes may serve to order the sequence reads from individual fragments as well as to differentiate between fragments with differing genetic origins (e.g., chromosomes). By counting the number of sequences mapped to each chromosome, the over- or underrepresentation of any chromosome in maternal plasma contributed by an aneuploid fetus is then detected.

c. Forensic Applications

DNA profiling (also called DNA testing, DNA typing, or genetic fingerprinting) is a technique employed by forensic scientists to assist in the identification of individuals by their respective DNA profiles. DNA profiles are encrypted sets of letters that reflect a person's DNA makeup, which can also be used as the person's identifier. DNA profiling is used in, for example, parental testing and criminal investigation.

DNA profiling uses repetitive (“repeat”) sequences that are highly variable called variable number tandem repeats (VNTRs), in particular short tandem repeats (STRs). VNTR loci are very similar between closely related humans, but are so variable that unrelated individuals are extremely unlikely to have the same VNTRs. However, traditional methods fail to provide consistent and reliable results since almost 99.9% of human DNA sequences are the same in every person, and most importantly, the target DNA is often contaminated by a large amount of foreign substances (e.g., environmental contaminations, victim vs. perpetrator cells and/or nucleic acids).

The methods, compositions and systems described herein may be applicable to identifying specific DNA sample in forensic analysis, by allowing characterization of minority represented nucleic acids in larger nucleic acid samples.

As described elsewhere herein, genetic material (e.g., DNA) may be extracted from a mix of forensic evidence (e.g., a mix of bloodstains, tissue, etc.). The extracted DNA samples and a plurality of beads carrying functional oligonucleotides are then co-partitioned into multiple reaction volumes or partitions via a controlled process such that each partition may comprise only a small number of beads and small amount of DNA samples. By providing the sample materials in the partitions at a level whereby each partition is unlikely to include overlapping sequences or segments of genomic material from different organisms (e.g., victim vs. perpetrator), one can ensure the processing and detection of the separate contributing sample nucleic acids, as well as attribution of such sample nucleic acids as between two different origins.

Oligonucleotides attached to beads may comprise a common sequence (e.g. a barcode sequence) and a prime sequence (a target N-mers targeting a specific region of DNA in current case). The common barcode sequences are used to identify samples and prime specific regions of sample DNA within each given partition. The initial amplification process may occur within each partition to generate amplified barcoded sequences. The amplicons may then be pooled and subjected to one or more additional amplification processes, followed by sequencing of the final amplified product. As described elsewhere herein, Barcode sequences included in amplicons may be used to attribute DNA sequences to their respective origins. By analyzing VNTR, in particular STR loci of amplified sequences, the subject that target DNA belongs to may be identified.

d. Environmental Testing

As with forensic testing described above, testing of environmental samples often involves looking for specific biological organisms or components within highly heterogeneous samples, e.g., containing large numbers of differing organisms, biological components, and other materials. In such cases, the methods and systems descried herein provide advantageous characterization of the various contributing components to a sample, e.g., through nucleic acid sequencing, without majority components overwhelming the analysis. Such analyses may include interrogation of samples for particular pathogens, indicator organisms, e.g., coliforms, and the like.

e. Microbiome Characterization

The compositions and methods descried herein may be useful in characterization of multiple individual population components, e.g., microbiome analysis, where the contribution of individual population members may not otherwise be readily identified amidst a large and diverse population of microbial elements. In particular, typical ensemble based sequencing approaches may tend to give an average or consensus of the overall genetic information from a mixed sample population, such that subtle variations in the genetic makeup as between members of the population will not be seen. Such variations can define differing strains, variants or species of microbiome members that ae important in characterizing the state of the given population or microbiome.

In an exemplary process, genetic material (e.g., DNA, RNA, etc.) extracted from a population of cells, e.g., a microbiome sample, may be partitioned into separate partitions (e.g., droplets), such that a partition is unlikely to include overlapping portions of nucleic acids from different members of the starting population. In some cases, this is accomplished by providing the nucleic acids extracted from the population at a concentration whereby the probability of such overlapping sequences being co-partitioned is very low. In some aspects, this may be accomplished by partitioning whole cells, such that individual cells are separately partitioned and processed as described herein, to characterize their nucleic acids. The beads with releasably attached oligonucleotides may be partitioned into the same sets of partitions. Again, the partitioning process may be controlled (e.g., controlled flow rate of sample stream, controlled flow rate of bead stream, controlled flow rates of both sample and bead stream, defined structure of geometry of channel junction, etc.) such that each partition may be occupied by certain numbers of beads or target nucleic acids, as described above.

Within each partition, sample may be initially amplified with the released oligonucleotides which include a common region (e.g., a barcode sequence) and a variable region (e.g., target N-mers or random N-mers). After this initial amplification process, amplified sequences within each individual partition may be tagged with a unique identifier (i.e., barcode sequence) which may attribute the resulting sequences to their respective partitions during the following, for example, sequencing process. In cases where the sample is allocated to that partition based upon its sample origin, the processing steps to which it is subsequently exposed, one can better identify the resulting sequences as having originated from a specific sample.

The amplicons may then be pooled and may be subjected to one or more additional amplification processes, followed by sequencing of the final amplified product. Based upon the unique barcode sequence attached, the sample origin of each resulting sequence may be identified.

### VIII. FILTERING OF CONTAMINATION

Contamination of a nucleic acid sample with non-sample nucleic acids can result in the random generation of extraneous sequencing reads that can complicate sequencing data analysis, including introducing errors into such analysis (e.g., sequence assembly). Nucleic acid contamination can generally be regarded as nucleic acid not derived from a nucleic acid sample of interest (e.g., “junk” nucleic acid). In some cases, such contamination is present at relatively low-levels, yet can still have an impact on the quality and accuracy of a sequence analysis.

Methods, compositions and systems described herein can be useful in identifying sequencing reads (e.g., a sequences determined for a barcoded fragment of a nucleic acid or a copy thereof) generated from nucleic acid contamination, including such contamination at relatively low-levels. In some cases, methods, systems and compositions described herein can be used to filter out nucleic acid (e.g., DNA) sequencing reads derived from contamination nucleic acid by one or more of identification and removal of the contaminating sequencing reads or by eliminating unidentifiable sequencing reads from identifiable sequencing reads when such nucleic acid contamination is present at relatively low levels, such as at less than 50%, less than 45%, less than 40%, less than 35%, less than 30%, less than 25%, less than 20%, less than 15%, less than 10%, less than 1%, less than 0.1%, less than 0.01%, less than 0.001%, less than 0.0001% or less than 0.00001% of the total nucleic acids in the sample.

In one aspect, the disclosure provides a method for analyzing a nucleic acid sequence. The method includes providing partitions (e.g., wells, tubes, micro or nanowells, through holes, fluid droplets (e.g., aqueous droplets within a water-in-oil emulsion)) comprising nucleic acid molecules generated from a nucleic acid sample. The nucleic acid molecules can be pooled from the partitions into a nucleic acid mixture that can then be subjected to nucleic acid sequencing to generate sequencing reads comprising nucleic acid sequences of the nucleic acid molecules. Using a programmed computer processor (e.g., such as a programmed computer processor of an example computer control system described herein), the sequencing reads can be analyzed and, when present, at least one contaminant read (e.g., associated with a contaminant nucleic acid molecule in the nucleic acid mixture) can be identified. Once identified, the contaminant read can be removed from the sequencing reads with a sequence of the nucleic acid sample generated from the remaining sequencing reads. In some cases, a plurality of contaminant reads (e.g., associated with the same contaminant nucleic acid molecule or associated with different contaminant nucleic acid molecules) are identified and removed prior to generating a sequence for the nucleic acid sample.

As is discussed above, the amount of the contaminant nucleic acid molecule in the nucleic acid mixture may be relatively low compared with the total amount of nucleic acid molecules in the nucleic acid mixture. For example, the amount of the contaminant nucleic acid molecule in the nucleic acid mixture may be less than 50%, less than 45%, less than 40%, less than 35%, less than 30%, less than 25%, less than 20%, less than 15%, less than 10%, less than 5%, less than 1%, less than 0.5%, less than 0.1%, less than 0.05%, less than 0.01%, less than 0.005%, less than 0.001%, less than 0.005%, less than 0.001%, less than 0.0005%, less than 0.0001%, less than 0.00005%, less than 0.00001%, less than 0.000005%, less than 0.000001%, less than 0.0000005%, less than 0.0000001%, or less of the total amount of nucleic acid molecules in the nucleic acid mixture.

In some embodiments, the contaminant read can be identified by determining sequence overlap(s) among subsets of the sequencing reads and identifying the contaminant read if overlap(s) for a given one of the sequencing reads is less than a threshold value with respect to all of the subsets. In some embodiments, the contaminant read can be identified by determining sequence overlap(s) among subsets of the sequencing reads and identifying the contaminant read if overlap(s) for a given one of the sequencing reads is less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, less than 0.001%, less than 0.0005%, less than 0.0001% or less with respect to all of the subsets. In some embodiments, the contaminant read can be identified by determining sequence overlap(s) among subsets of the sequencing reads and identifying the contaminant read if a given one of the sequence reads does not overlap with respect to all of the subsets.

In some embodiments, the contaminant read can be identified by comparing the sequence reads to a reference and identifying a given sequence read of the sequence reads as the contaminant read if the given sequencing read overlaps with the reference at less than a threshold value. In some embodiments, the contaminant read can be identified by comparing the sequence reads to a reference and identifying a given sequence read of the sequence reads as the contaminant read if the given sequencing read overlaps with the reference at less than 50%, at less than 45%, at less than 40%, at less than 35%, at less than 30%, at less than 25%, at less than 20%, at less than 15%, at less than 10%, at less than 9%, at less than 8%, at less than 7%, at less than 6%, at less than 5%, at less than 4%, at less than 3%, at less than 2%, at less than 1%, at less than 0.5%, at less than 0.1%, at less than 0.05%, at less than 0.01%, at less than 0.005%, at less than 0.001%, at less than 0.0005%, at less than 0.0001% or less. In some embodiments, the contaminant read can be identified by comparing the sequence reads to a reference and identifying the contaminant read if a given one of the sequence reads does not overlap with the reference.

In some embodiments, the contaminant read can be identified by comparing the sequence reads to one another to identify sequence overlap(s) among the sequencing reads and identifying a given sequence read of the sequence reads as the contaminant read if its sequence overlap with other sequencing reads among the sequencing reads is less than a threshold value. In some embodiments, the contaminant read can be identified by comparing the sequence reads to one another to identify sequence overlap(s) among the sequencing reads and identifying a given sequence read of the sequence reads as the contaminant read if its sequence overlap with other sequencing reads among the sequencing reads is less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, less than 0.001%, less than 0.0005%, less than 0.0001% or less. In some embodiments, the contaminant read can be identified by comparing the sequence reads to one another to identify sequence overlap(s) among the sequencing reads and identifying a given sequence read of the sequence reads as the contaminant read if its sequence does not overlap with a sequence of the other sequencing reads among the sequencing reads.

In some embodiments, the contaminant read can be identified by mapping the sequence reads to their respective sequence region(s) and identifying a given sequence read of the sequence reads as the contaminant read if, when mapped to its sequence region(s), the given sequence read overlaps with less than a threshold number of the other sequence reads when mapped to their sequence region(s). In some embodiments, the contaminant read can be can be identified by mapping the sequence reads to their respective sequences and identifying a given sequence read of the sequence reads as the contaminant read if, when mapped to its sequence region(s), the given sequence read overlaps with less than 50 other reads of the sequence reads, less than 45 other reads of the sequence reads, less than 40 other reads of the sequence reads, less than 35 other reads of the sequence reads, less than 30 other reads of the sequence reads, less than 25 other reads of the sequence reads, less than 20 other reads of the sequence reads, less than 19 other reads of the sequence reads, less than 18 other reads of the sequence reads, less than 17 other reads of the sequence reads, less than 16 other reads of the sequence reads, less than 15 other reads of the sequence reads, less than 14 other reads of the sequence reads, less than 13 other reads of the sequence reads, less than 12 other reads of the sequence reads, less than 11 other reads of the sequence reads, less than 10 other reads of the sequence reads, less than 9 other reads of the sequence reads, less than 8 other reads of the sequence reads, less than 7 other reads of the sequence reads, less than 6 other reads of the sequence reads, less than 5 other reads of the sequence reads, less than 4 other reads of the sequence reads, less than 3 other reads of the sequence reads, less than 2 other reads of the sequence reads, less than 1 other read of the sequence reads or with none of the other reads of the sequence reads when mapped to their sequence region(s).

As described elsewhere herein, a nucleic acid sample can be fragmented and the fragments partitioned, such as, for example into droplets of an emulsion (e.g., as shown in FIG. 4). In each droplet, barcoded fragments or copies thereof of the partitioned fragments can be generated, such as, for example, in an amplification reaction with respect to FIG. 3 and as is described elsewhere herein. The barcoded fragments or copies thereof can then be sequenced to generate barcoded fragment reads, which can then be assembled into larger sequences. Where a contaminant nucleic acid molecule(s) is present in the nucleic acid sample and/or a partition in which barcoded fragments are generated, barcoded fragments or copies thereof corresponding to the contaminant nucleic acid molecule(s) can also be generated. Such contaminant barcoded fragments or copies thereof can be also be sequenced, thus, introducing extraneous sequencing reads into a sequence analysis. Such extraneous sequencing reads can interfere with and/or introduce error into a sequence analysis of the nucleic acid sample. The methods provided herein can be useful for removing barcoded reads generated from barcoded fragments or copies thereof that are derived from a contaminant nucleic acid molecule. Accordingly, in some embodiments, providing partitions comprising nucleic acid molecules generated from a nucleic acid sample can include generating barcoded fragments or copies thereof that correspond to each of the nucleic acid molecules, such as, for example by methods described herein. Moreover, the sequencing reads that are generated can include barcoded fragment reads comprising nucleic acid sequences of the barcoded fragments or copies thereof.

In the case where the nucleic acid sample is a genomic nucleic acid sample, a lack of overlap of a sequence read to another sequence read comprising a sequence of a known neighboring portion of the genome (e.g., mappability to a common known or predominant sequence) can be used to identify the sequence read as the contaminant sequence read. In some cases, though, it is possible for a sequencing read not to be linked to a known neighboring portion of a genome, yet still map to sequence regions that are linked (e.g., as evidenced by significant barcode overlap between the sequence regions), such as in the case of structural variants (e.g., copy number variation, an insertion, a deletion, a translocation, an inversion, a rearrangement, a repeat expansion, a duplication) or other genetic variations (e.g., single nucleotide polymorphisms). Example methods and systems for determining structural variants and other genetic variations are provided in e.g., U.S. Provisional Patent Application No. 62/017,808, filed Jun. 26, 2014 and U.S. Provisional Patent Application No. 62/072,214, filed Oct. 29, 2014 each of which applications is herein incorporated by reference in its entirety for all purposes.

Accordingly, an appropriate threshold value for common barcode sequences between sequence regions to which a given sequence read maps can be set in order to identify a given sequence read as the contaminating read, where it is not otherwise linked to a known neighboring portion of the genome. For example, the contaminant read can be identified by identifying a given one of the barcoded fragment reads as the contaminant read if sequence regions to which the given barcoded fragment read maps map barcoded fragments having common barcode sequences between the sequence regions of less than 50%, less than 45%, less than 40%, less than 35%, less than 30%, less than 25%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.5%, less than 0.1%, less than 0.05%, less than 0.01%, less than 0.005%, less than 0.001%, less than 0.0005%, less than 0.0001%, or even less of the total barcoded fragment reads mappable to the sequence regions.

Removing contaminant reads from sequence construction can result in improved accuracy in generating the sequence of the nucleic acid sample. For example, by identifying the contaminant read and removing it from generating the sequence of the nucleic acid sample, the sequence can be generated at an accuracy of at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.9%, at least 99.99%, at least 99.999%, at least 99.9999% or higher.

### IX. COMPUTER CONTROL SYSTEMS

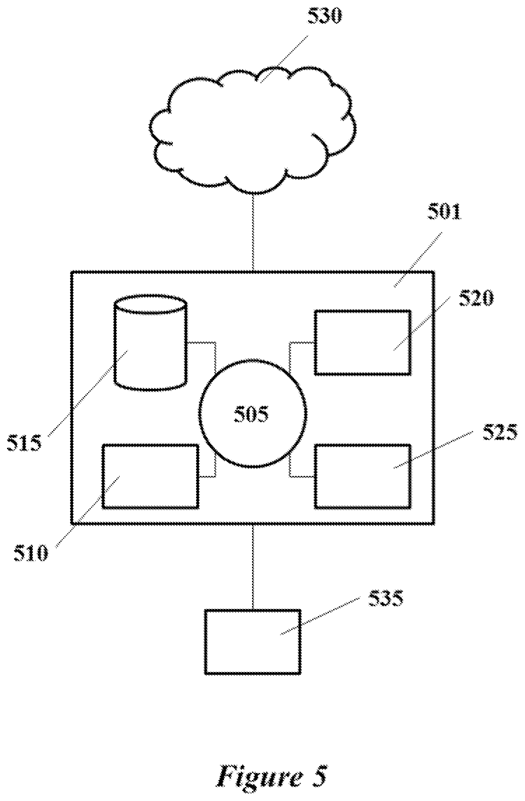

The present disclosure provides computer systems that are programmed or otherwise configured to implement methods provided herein, such as, for example, methods for nucleic sequencing (e.g., nucleic acid sequencing of a low input/low amount of nucleic acid), analysis and interpretation of obtained sequencing data (e.g., including in applications described herein such as in detecting an diagnosing disease, in identification of fetal aneuploidy, in forensic applications, in microbiome characterization, in environmental testing), and/or identifying and filtering of contaminating sequencing reads prior to or during sequence assembly. An example of such a computer system is shown in FIG. 5. As shown in FIG. 5, the computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. Examples of operations performed by the CPU 505 can include fetch, decode, execute, and writeback. The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet. The computer system 501 can communicate with one or more remote computer systems through the network 530. For instance, the computer system 501 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510. The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that can comprise a user interface (UI) for providing, for example, an output or readout of a nucleic acid sequencing instrument coupled to the computer system 501. Such readout can include a nucleic acid sequencing readout, such as a sequence of nucleic acid bases of a given nucleic acid sample. The UI may also be used to display the results of an analysis making use of such readouts and any statistical data accompanying such an analysis. Examples of UI's include, without limitation, a graphical user interface (GUI) and web-based user interface. The electronic display 535 can be a computer monitor, or a capacitive or resistive touchscreen.

### X. EXAMPLES

### Example 1: Screening for Aneuploidy by Analyzing Cell-free Fetal DNA

A blood sample containing less than 8% cell-free fetal DNA is taken from a pregnant woman. Cell-free plasma DNA extracted from the blood sample. The extracted cell-free DNA samples are then co-partitioned with beads attached to releasably functional oligonucleotides into multiple droplets. Within each droplet, DNA samples are amplified by released oligonucleotides. The amplicons are then pooled and subjected to an additional amplification process, followed by analysis and sequencing of the amplified product. The unique barcode attached to DNA samples within partitions enables the attribution of resulting sequences to their respective genetic origins (e.g., chromosomes). By counting the number of sequences mapped to each chromosome, the over-or underrepresentation of any chromosome in maternal plasma contributed by an aneuploid fetus is then detected.

### Example 2: Monitoring Metastatic Progression in Cancer Patient by Detecting Circulating Tumor-Associated DNA

A blood sample comprising less than 1% circulating tumor cells is collected from a patient with metastatic prostate cancer and plasma DNA is isolated from the blood sample. The extracted DNA sample is then partitioned into a plurality of the reaction volumes or partitions with a predetermined sample/partition ratio such that each partition contains no more than one individual target DNA. The partitioned DNA sample is then subjected to several processing steps including: (1) partitioning a plurality of beads with releasably connected oligonucleotide tags into the partition to form a sample-bead mixture, (2) releasing the functional oligonucleotides including a barcode sequence and a random N-mer sequence into the partition, (3) amplifying the sample with the random N-mer within each partition, and (4) sequencing the amplicons and analyzing the sequence read based upon, the unique barcode sequence included in each amplicon. The concentration of circulating tumor-associated DNA in the blood of tumor patient is then compared with those of controls. A rising circulating tumor-associated DNA yields signals the further progression of the cancer.

### Example 3: Analyzing a Large Collection of Environmental Bacterial Isolates by Ribosomal DNA Sequencing

A collection of bacterial isolates is taken from environmental sources and tested. DNA is extracted from each isolate and partitioned into multiple reaction volumes or partitions such that each partition contains DNA sample originating from a specific bacterial isolate. A plurality of beads attached with functional oligonucleotides which include a unique barcode sequence and a 16s rDNA primer is then added into partitions to form a mixture with DNA samples within each partition. Extracted DNA sample in each partition is then amplified with the universal 16s rDNA primer. The amplified product is then sequenced and compared with those available in the database. Identification to the species level is defined as a sequence similarity of ≥99% with that of the prototype strain sequence in the database, and identification at the genus level is defined as a sequence similarity of >97% with that of the prototype strain sequence in the database. Using the sequencing information, the percentage of each strain within the collection of bacterial isolates is determined.

### Example 4: Analyzing Cellular Nucleic Acids

Genomic DNA is extracted from multiple cell lines (NA12878, NA12877, NA12882, NA20847) using Qiagen High Molecular Weight MagAttract DNA Kit. Genomic DNA is quantified using the Qubit system and titrated down to concentrations so as to partition three different starting masses of DNA into droplets of an emulsion: 2.4 ng, 1.2 ng or 0.6 ng along with barcoded beads. Barcoded sequencing libraries are prepared in emulsion droplets in a manner analogous to that shown in FIG. 4 and described elsewhere herein, the emulsion broken and the droplet contents pooled and the sequencing libraries enriched by hybrid capture using Agilent SureSelect Target Enrichment (Human V5). Libraries are sequenced to ˜160× on-target sequencing depth. Variant-calling is performed using Long Ranger software. Briefly, sequencing reads are aligned using BWA MEM, sorted by position, marked for PCR duplicates, and the Freebayes software package is then used to called SNPs, small insertions and deletions. Samples are characterized against previously established ground truths for sensitivity and positive predictive value (PPV) of SNPs, insertions and deletions. For SNPs, sensitivity and PPV are both >95%, for insertions and deletions, PPV is >90% and sensitivity is >70%.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-120. (canceled)

121. A method of processing nucleic acid molecules, comprising:
(a) providing a plurality of nucleic acid molecules, wherein said plurality of nucleic acid molecules comprises less than about 50 nanograms (ng) of nucleic acid molecules;
(b) partitioning said plurality of nucleic acid molecules with a plurality of beads into a plurality of partitions, wherein a partition of said plurality of partitions comprises a nucleic acid molecule of said plurality of nucleic acid molecules and a bead of said plurality of beads, wherein said bead comprises a plurality of oligonucleotides coupled thereto, wherein an oligonucleotide of said plurality of oligonucleotides comprises a barcode sequence; and;
(c) within said partition:
(i) subjecting said bead to conditions sufficient to release said oligonucleotide from said bead; and
(ii) using said oligonucleotide released from said bead and said nucleic acid molecule to generate a nucleic acid product, wherein said nucleic acid product comprises said barcode sequence, or complement thereof, and a sequence of said nucleic acid molecule, or complement thereof.

122. The method of claim 121, further comprising recovering said nucleic acid product, or derivative thereof, from said partition.

123. The method of claim 121, further comprising identifying (i) said barcode sequence, or complement thereof, and (ii) said sequence of said nucleic acid molecule.

124. The method of claim 123, further comprising detecting a single nucleotide polymorphism, an insertion, or a deletion of said nucleic acid molecule, wherein said nucleic acid molecule comprises genomic deoxyribonucleic acid.

125. The method of claim 123, wherein the sensitivity for insertions and deletions is at least 70%, the positive predictive value of insertions and deletions is at least 90%, the sensitivity of single nucleotide polymorphisms is at least 95%, or the positive predictive value of single nucleotide polymorphisms is at least 95%.

126. The method of claim 121, wherein subjecting said bead to conditions sufficient to release said oligonucleotide from said bead comprises subjecting said bead to conditions sufficient to at least partially degrade said bead.

127. The method of claim 121, wherein subjecting said bead to conditions sufficient to release said oligonucleotide from said bead comprises applying a stimulus to said bead.

128. The method of claim 127, wherein said stimulus comprises a temperature change, a pH change, light, a chemical species, a reducing agent, or any combination thereof.

129. The method of claim 121, wherein said plurality of nucleic acid molecules comprises less than about 20 ng of nucleic acid molecules.

130. The method of claim 129, wherein said plurality of nucleic acid molecules comprises less than about 1 ng of nucleic acid molecules.

131. The method of claim 121, wherein said oligonucleotide comprises a primer sequence.

132. The method of claim 131, wherein said primer sequence comprises a random N-mer sequence.

133. The method of claim 131, wherein said primer sequence comprises a targeted primer sequence.

134. The method of claim 121, wherein (c) comprises performing a primer extension reaction.

135. The method of claim 121, wherein (c) comprises performing a ligation reaction.

136. The method of claim 121, wherein said barcode sequence comprises 6-20 nucleotides.

137. The method of claim 121, wherein said plurality of partitions is a plurality of wells.

138. The method of claim 121, wherein said plurality of partitions is a plurality of droplets.

139. The method of claim 121, wherein said plurality of nucleic acid molecules is derived from a bodily fluid.

140. The method of claim 121, wherein said plurality of nucleic acid molecules comprises nucleic acid molecules derived from circulating tumor nucleic acid.

141. The method of claim 121, wherein said plurality of nucleic acid molecules comprises nucleic acid molecules derived from fetal nucleic acid.
